(19) United States
(12) Patent Application Publication
(10) Pub. No.: US 2018/0282424 A1
(43) Pub. Date: Oct. 4, 2018

(54) MOLECULES WITH BIMODAL ACTIVITY DEPLETING TARGET AT LOW DOSE AND INCREASING IMMUNOSUPPRESSION AT HIGHER DOSE

(71) Applicants: GLIKNIK INC., Baltimore, MD (US); UNIVERSITY OF MARYLAND, BALTIMORE, Baltimore, MD (US)

(72) Inventors: David S. BLOCK, Baltimore, MD (US); Henrik OLSEN, Baltimore, MD (US); Scott STROME, Reisterstown, MD (US); Xiaoyu ZHANG, Catonsville, MD (US)

(21) Appl. No.: 15/524,883

(22) PCT Filed: Nov. 6, 2015
(86) PCT No.: PCT/US15/59574

§ 371 Date: May 5, 2017

Related U.S. Application Data
(60) Provisional application No. 62/076,378, filed on Nov. 6, 2014.

(51) Int. Cl. C07K 16/28

(52) U.S. Cl. CPC C07K 16/2887; C07K 2317/55; A61K 2039/545; C07K 2317/53; C07K 2317/73; C07K 2317/52

(57) ABSTRACT

The present disclosure involves biologically active proteins termed stradobodies and having bimodal activity. Thus, the present disclosure provides compositions and methods providing both target cell destructive and immunosuppressive activities, useful in the treatment of diseases and conditions including autoimmune diseases, inflammatory diseases, infectious diseases, or cancers.

### CROSS REFERENCE TO RELATED APPLICATIONS

This application claims priority to U.S. Provisional Application No. 62/076,378, filed Nov. 6, 2014, which is incorporated herein by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GLIK-014_01WO_SeqList.txt; date recorded: Nov. 5, 2015, file size 411 kilobytes).

### BACKGROUND

Monoclonal antibody (mAb) therapy is an important and growing part of medicine. Over 30 monoclonal antibodies have been approved for various autoimmune diseases, inflammatory diseases, infectious diseases, and cancers in the United States, Europe, and elsewhere with hundreds more under investigation. Many antibodies are in development or are used clinically for the purpose of therapeutically depleting target cells. However, common problems in monoclonal antibody therapy development include a lack of potency, loss of efficacy, and unwanted pro-inflammatory effects that can occur upon depletion of target cells. For example, the chimeric α-CD20 antibody, rituximab, is approved for the treatment of four types of CD20 positive non-Hodgkin's Lymphoma (NHL), CD20 positive chronic lymphocytic leukemia (CLL), granulomatosis with polyangiitis, microscopic polyangiitis, and rheumatoid arthritis that is refractory to TNF blockade. However, in people rituximab is dosed weekly at 375 mg/m² or greater which is approximately 8.1 mg/Kg per week. Moreover, in many patients, rituximab therapy is not effective or loses efficacy over time, or elicits unwanted inflammation that may be associated with chest pain, irregular heartbeat, kidney damage, bowel perforation, and other serious medical problems.

As another example, monoclonal antibodies may have some efficacy in viral, bacterial, and fungal infectious disease treatment, but fail to ameliorate, or may even exacerbate, the inflammatory and immunopathogenic effects elicited by the infection.

Many infectious diseases are characterized by both infection and by a host inflammatory reaction that may be detrimental to the patient. With many infections, including viral infections such as Ebola, there is a need to target the infectious agent for depletion and to modulate the body's potentially fatal inflammatory response to the infection.

Thus, there is a need for new antibody-based therapeutics in the treatment of autoimmune disorders, inflammatory diseases, infectious diseases, and cancers that exhibit an optimal level of target cell depletion while avoiding the detrimental effects of unwanted inflammation.

### SUMMARY OF THE INVENTION

In one aspect, the present disclosure provides compositions and methods for inducing target destruction (e.g., target cell lysis, target cell depletion, or target antigen destruction) and immune suppression or tolerance at two different doses. In another aspect, the present disclosure provides compositions and methods for inducing target cell destruction (e.g., target cell lysis, target cell depletion, or target antigen destruction) and immune suppression or tolerance, wherein the different effects are derived from different relative amounts of homodimeric or multimeric forms of the composition (e.g., homodimer or lower order multimers being associated with cell lysis and higher order multimers being associated with immune tolerance). Thus, the compositions and methods provide a bimodal activity useful in the treatment of diseases and conditions including autoimmune diseases, inflammatory diseases, alloimmunization diseases, infectious diseases, or cancers. The compositions and methods provide improved means of antibody-based target cell lysis and target cell depletion coupled with the advantage of controlling the level of inflammation present in the subject, which may be due to the disease or condition, or to the administration of an antibody-based therapy, or to a combination thereof.

In one aspect, target cell depletion and immune suppression are achieved using a stradobody. In one embodiment, the stradobodies provided herein comprise an Fab domain that is specific for a target antigen. In another embodiment, the stradobodies provided herein are multimerizing stradobodies. In one aspect, the stradobodies provided herein exhibit a bimodal activity, wherein the stradobodies are capable of antibody-like target cell depletion activity as well as human Intravenous Immunoglobulin (“IVIG”)-like immune suppression. Thus, in one aspect, the present disclosure provides methods for eliciting antibody-like target cell depletion and IVIG-like immune suppression in a subject by administering to the subject a stradobody. Such antibody-like target cell depletion may involve the effector functions Complement Dependent Cytotoxicity, Antibody Dependent Cellular Cytotoxicity, Antibody Dependent Cellular Phagocytosis, Fc-dependent apoptosis, and/or additional mechanisms.

In one aspect, the stradobodies provided herein exhibit a bimodal activity, wherein the activity is determined by the level of target antigen present in the subject. Thus, in one aspect, the stradobodies provided herein exhibit antigen-dependent bimodal activity. In one embodiment, the stradobodies are capable of antibody-like target depletion activity, and are further capable of IVIG-like immune suppression after optimal target depletion or when no target antigen is present.

In another aspect, the bimodal activity of the stradobodies provided herein is achieved by administering to a subject different dosing levels of stradobodies. Thus, one aspect, the stradobodies provided herein exhibit dose-dependent bimodal activity. In one embodiment, the stradobodies exhibit antibody-like target cell depletion activity when the stradobodies are present at low concentrations and exhibit IVIG-like immune suppression when the stradobodies are present at higher concentrations.

In another aspect, the stradobodies provided herein exhibit a bimodal activity, wherein the activity is determined by the amount of homodimeric or multimeric forms of the stradobodies comprising the compositions. In one embodiment, the stradobodies that are homodimers or lower order multimers such as the dimer of the homodimer are capable of antibody-like target depletion activity and the stradobodies that are higher order multimers (for example, the tetramer, pentamer, and hexamer of the homodimer) are capable of IVIG-like immune suppression. In some embodiments, the stradobodies capable of antibody-like target depletion activity are comprised of more than about more than about 50%, more than about 60%, more than about 70%, more than about 80%, or more multimer bands that are lower order multimers (e.g., the homodimer and the dimer of the homodimer). In some embodiments, the stradobodies capable of IVIG-like immune suppression are comprised of more than about 50%, more than about 60%, more than about 70%, more than about 80%, or more multimer bands at higher orders than the homodimer and dimer of the homodimer.

In one aspect, the stradobodies provided herein exhibit similar or increased target depletion, similar or increased duration of target depletion, and/or similar or more specific target depletion relative to a monoclonal antibody comprising the identical Fab region. Such target cell depletion can be, without limitation, B cells or other host immune cells, viruses or other infectious agents, or any cancer cell. In another aspect, the stradobodies provided herein exhibit increased immune suppression relative to a monoclonal antibody comprising the identical Fab region.

In one aspect, the stradobodies provided herein present multivalent Fab′ to an antigen and multivalent Fc to immune cells. In another aspect, at low doses of stradobody the binding of the multivalent Fab′ of the stradobodies provided herein to the target cell antigen outcompetes the binding of the multivalent Fc binding of the stradobodies provided herein to immune cells, resulting in relatively more target-directed cell killing than target-directed tolerance. In another aspect, at high doses of stradobody, the binding of the multivalent Fc of the stradobodies provided herein to immune effector cells outcompetes the binding of the multivalent Fab′ of the stradobodies provided herein to immune cells, resulting in relatively more target-directed immune tolerance than target-directed cell killing. In another aspect, with doses of stradobody homodimer and dimer of the homodimer, the binding of the multivalent Fab′ of the stradobodies provided herein to the target cell antigen outcompetes the binding of the Fc binding of the stradobodies provided herein to immune cells, resulting in relatively more target-directed cell killing than target-directed tolerance, similar to a monoclonal antibody. In another aspect, with doses of stradobody higher order multimers, the binding of the multivalent Fc of the stradobodies provided herein to immune effector cells outcompetes the binding of the multivalent Fab′ of the stradobodies provided herein to immune cells, resulting in relatively more target-directed immune tolerance than target-directed cell killing.

In one aspect, the present disclosure provides the surprising finding that a stradobody, despite having antibody-like features such as comprising an Fab and an Fc, may be used to induce immune suppression. In some embodiments, immune suppression is achieved in a subject following stradobody-mediated depletion of a target cell. This may be achieved through continuous dosing of the depleting dose of the stradobody provided herein, or may be achieved by use of a higher dose, or may be achieved by use of a lower order multimer followed by a higher order multimer of the stradobody or by use of a low dose followed by a high dose. In other embodiments, the immune-suppressive effects of a stradobody are achieved at a high concentration or high dose level of stradobody, such that the Fc binding activity of the stradomer portion of a stradobody elicits immune suppressive effects. In one embodiment, the stradobody is present at an in vitro concentration that is more than about 0.1 μg/mL, more than about 0.5 μg/mL, more than about 1 μg/mL, more than about 2.5 μg/mL, more than about 5 μg/mL, more than about 10 μg/mL, more than about 20 μg/mL, more than about 50 μg/mL, or more than about 100 μg/mL. In one embodiment, the stradobody is present at an in vitro concentration that is more than 1 μg/mL. In another embodiment, the in vivo dosing level is determined based on the effects of the in vitro concentration of the stradobody. For example, in one embodiment, an in vivo dosing level that achieves levels equivalent to the effective in vitro concentration are used to achieve immune-suppressive effects in vivo. In another embodiment, the immune-suppressive effects of a stradobody are achieved in a subject by administering to the subject a stradobody at a dosing level of more than about 0.1 mg/kg, more than about 0.5 mg/kg, more than about 1 mg/kg, more than about 2.5 mg/kg, more than about 5 mg/kg, more than about 10 mg/kg, more than about 20 mg/kg, more than about 50 mg/kg, or more than about 100 mg/kg. In one embodiment, the immune-suppressive effect of the stradobody is achieved in a subject by administering to the subject a stradobody at a dosing level of more than 1 mg/kg. In one embodiment, the dose required to induce tolerance is less when administering higher order multimers of the stradobody relative to the same stradobody comprising high amount of the homodimer and dimer of the homodimer.

In one aspect, the present disclosure provides methods for inducing target cell depletion followed by suppression of inflammation in a subject, the method comprising administering a stradobody comprising an Fab domain that is specific for a target antigen expressed on a target cell in the subject. In one embodiment, administration of the stradobody induces optimal target cell depletion. In a further embodiment, optimal target cell depletion is achieved when the presence of target cells expressing the target antigen in the subject has reached low or absent levels. In a yet further embodiment, the induction of immune suppression occurs after target cell depletion has occurred due to a lack of target antigen, such that in the absence of target antigen, the Fc binding activity of the multimeric Fc stradomer portion of a stradobody elicits IVIG-like immune suppressive effects.

In one aspect, the present disclosure provides methods for inducing target cell depletion followed by suppression of inflammation in a subject, the method comprising administering a stradobody at a first dose level followed by a second dose level. In a further embodiment, the second dose level is higher than the first dose level. In a still further embodiment, the first dose level results in more target cell depletion than IVIG-like immune suppression, and the second dose level results in more IVIG-like suppression of inflammation than target cell depletion in the subject. In another aspect, the present disclosure provides methods for inducing target cell depletion followed by suppression of inflammation in a subject, the method comprising administering first either a monoclonal antibody or the stradobody comprising the same Fab with higher ratios of homodimer and dimer followed by a second dose of the full stradobody or the stradobody with higher ratios of the higher order multimers. In a further embodiment, the higher order multimers of the second and subsequent doses are the tetramer, pentamer, hexamer and other higher order multimers of the homodimer. In a still further embodiment, the first dose results in more target cell depletion than IVIG-like immune suppression and the second dose level results in more IVIG-like suppression of inflammation in the subject than target cell depletion.

In one aspect, the present disclosure provides methods for inducing target cell depletion followed by suppression of inflammation in a subject, the method comprising administering a monoclonal antibody followed by administering a stradomer. In another aspect, the present disclosure provides methods for inducing target cell depletion followed by suppression of inflammation in a subject, the method comprising administering a stradobody that has a higher ratio of homodimer and dimer compared with the native stradobody, followed by administering a stradomer. In another aspect, the present disclosure provides methods for inducing target cell depletion followed by suppression of inflammation in a subject, the method comprising administering a stradobody followed by administering a therapeutically effective amount of a stradomer.

In another aspect, the present disclosure provides methods for inducing target cell depletion followed by suppression of inflammation in a subject, the method comprising administering a stradobody at a first dose level followed by administering a therapeutically effective amount of IVIG. In a further embodiment, the first dose level of the stradobody is a low dose of stradobody. In a still further embodiment, the dose level of the stradobody has been manufactured to have a higher ratio of homodimer and dimer compared with the native stradobody produced from cells.

Thus, in one aspect, the present disclosure provides methods for inducing target cell depletion followed by suppression of inflammation in a subject, wherein the method comprises administering a stradobody, wherein the administration of a stradobody results in target cell depletion, and wherein administration of the stradobody is followed by administration of any of a) a second dose level of stradobody, wherein the second dose level is higher than the first dose level, b) a therapeutically effective amount of a stradomer, or c) a therapeutically effective amount of IVIG, in each case resulting in IVIG-like suppression of inflammation in the subject. The target cell depletion can be the result of administration of any of a) a lower dose of stradobody compared with subsequent doses, b) stradobody that has been manufactured to have a lower ratio of higher order multimers compared with the native stradobody, or c) even the monoclonal antibody comprising the same Fab as the stradobody.

In one embodiment, the stradobodies provided herein are administered at a first dose level followed by a second dose level. In one embodiment, the first dose level is less than about 10 mg/kg, less than about 5 mg/kg, less than about 1 mg/kg, less than about 0.5 mg/kg, less than about 0.1 mg/kg, less than about 0.05 mg/kg, or less than about 0.01 mg/kg. In one embodiment, the second dose level is more than about 0.1 mg/kg, more than about 0.5 mg/kg, more than about 1 mg/kg, more than about 2.5 mg/kg, more than about 5 mg/kg, more than about 10 mg/kg, more than about 20 mg/kg, more than about 50 mg/kg, or more than about 100 mg/kg. In one embodiment, the first dose level is about 0.1 mg/kg or about 1 mg/kg. In another embodiment, the second dose level is about 1 mg/kg or 10 mg/kg. In one embodiment, the first dose level is 0.01-1 mg/kg and the second dose level is 2.0-10 mg/kg. In a preferred embodiment, the first dose level is 1 mg/kg and the second dose level is 2.0-10 mg/kg.

In one aspect, the present disclosure provides methods for inducing target cell depletion followed by suppression of inflammation in a subject, the method comprising (i) administering a first multimerizing stradobody that is a homodimer or a lower order multimer, wherein the first multimerizing stradobody results in target cell depletion; and (ii) administering a second multimerizing stradobody, wherein the second multimerizing stradobody comprises a higher order multimer, and wherein the second multimerizing stradobody results in suppression of inflammation in the subject. In some embodiments, the first and second stradobody have the same structure and/or the same antibody specificity, and differ only in that the first stradobody is present predominantly as a homodimer or a lower order multimer and the second stradobody is present predominantly as a higher order multimer. In some embodiments, the first and second stradobody have the identical amino acid sequence, and differ only in that the first stradobody is present as a homodimer or a lower order multimer and the second stradobody is present as a higher order multimer.

In one embodiment, the subject is a human. In one embodiment, the suppression of inflammation is measured by a reduction in inflammatory cytokines such as, for example, IFNγ, TNF-α, IL-12, IL-6, and others known in the art; or increase in anti-inflammatory cytokines such as, for example, IL-1RA. In another embodiment, the suppression of inflammation in the subject is measured by changes in cell populations such as an increase in regulatory T cells and/or by changes in immune cell surface markers such as, for example, monocyte HLA-DR or B cell maturation markers, and/or changes in complement components detectable in serum. In one aspect, the present disclosure provides methods for treating a disease or condition in a subject in need thereof, the method comprising administering a stradobody to the subject. The stradobody, in one embodiment, comprises an Fab domain specific for a target antigen expressed on a target cell that is present in the subject. In one embodiment, the target antigen and/or target cell is associated with the disease or condition. For example, in one embodiment, the target antigen is CD20, EGFR, TNF-α, Rho(D), IL17, IL12/23, or Her2/neu. In some embodiments, the stradobodies are administered at different dose levels as disclosed herein, wherein a first dose level achieves optimal target cell depletion and a subsequent dose level induces immune suppression in the subject. In other embodiments, the administration of the stradobody at a single dose level results in target cell depletion in the subject followed by immune suppression. In other embodiments, the stradobodies are administered as a first stradobody that is primarily a homodimer or a lower order multimer, wherein the administration of the homodimer or lower order stradobody results in target cell depletion in the subject; and as a second stradobody that is primarily a higher order multimer, wherein the administration of the higher order stradobody results in immune suppression.

In some embodiments, the stradobody comprises an Fab domain specific for a target antigen on an infectious agent. Infectious agents include, without limitation, bacteria, viruses, fungi, and mycobacteria. Examples of viruses that can be targeted by the stradobody include an Fab directed against any of the viruses listed at http://en.wikipedia.org/wiki/List_of_viruses.

In one embodiment, the present disclosure provides methods for treating a subject having a disease caused by an infectious agent, the method comprising administering to the subject a stradobody, wherein the stradobody comprises an Fab specific for a target antigen on the infectious agent, and wherein the stradobody initially induces opsonization and destruction of the infectious agent, and wherein opsonization and destruction of the target antigen may be followed by immune suppression or immune tolerance in the subject. In another embodiment, the stradobody comprises an Fab domain specific for the infectious agent, and the stradobody is administered at different dose levels or is administered in different homodimeric or multimeric form, as disclosed herein. In one embodiment, at a first dose level, the stradobody binds the target antigen on the infectious agent and opsonizes it for destruction which may involve effector cell functions including CDC, ADCC, and/or ADCP; and at a second dose level, the stradobody binds Fc receptors (FcγRs) and induces immune suppression or immune tolerance in the subject which may involve suppression of effector functions including inhibition of CDC, ADCC, and/or ADCP. In a further embodiment, the second dose level is higher than the first dose level. In another embodiment, a first stradobody is predominantly a homodimer or lower order multimer and a second stradobody is predominantly a higher order multimer. In one embodiment, the induction of immune suppression or immune tolerance in the subject reduces or ameliorates the immunopathogenic effects of the infection. In one embodiment, the present disclosure provides methods for treating a subject having a disease caused by an infectious agent, the method comprising administering to the subject a stradobody, and wherein the method further comprises administering to the subject any of a) a second dose level of stradobody that is higher than the first dose level of stradobody, b) a therapeutically effective amount of a stradomer, or c) a therapeutically effective amount of IVIG, in each case resulting in destruction of the target infectious agent followed by IVIG-like suppression of inflammation in the subject. In another embodiment, the present disclosure provides methods for treating a subject having a disease caused by an infectious agent, the method comprising administering to the subject a stradobody that is predominantly a homodimer or a lower order multimer, and wherein the method further comprises administering to the subject a second stradobody that is predominantly a higher order multimer.

In one embodiment, the subject is a human.

In one aspect, the stradobodies having bimodal activity comprise an Fab domain, at least one multimerization domain, and at least one Fc domain. Thus, in some embodiments, the stradobodies are multimerizing stradobodies. In a further embodiment, the multimerization domain is selected from the group consisting of an IgG2 hinge, an isoleucine zipper, and a GPP repeat. In one embodiment, the stradobody comprises an Fab domain, an isoleucine zipper, and one or more Fc domains. In another embodiment, the stradobody comprises an Fab domain, an IgG2 hinge, and one or more Fc domains. In still another embodiment, the stradobody comprises an Fab domain, an isoleucine zipper, an IgG2 hinge, and one or more Fc domains. In some embodiments, the stradobody comprises two Fc domains. In one embodiment, the stradobody comprises at least one Fc domain, wherein the at least one Fc domain is an IgG1 Fc domain. In a further embodiment, the IgG1 Fc domain comprises an IgG1 hinge, IgG1 CH2, and IgG1 CH3. In one embodiment, the stradobody comprises an Fab domain that is specific for an antigen that is associated with a disease or condition. For example, in one embodiment, the stradobody comprises an Fab domain that is specific for TNF-α, Rho(D), IL-17, IL12/23, CD20, EGFR, or HER2/neu.

In one aspect, the present disclosure provides compositions and methods for treating cancer or an infectious disease in a subject, comprising administering a multimerizing stradobody to the subject, wherein the multimerizing stradobody is a homodimer or a lower order multimer, and wherein the multimerizing stradobody comprises an Fab domain specific for an antigen expressed on a tumor or a cancer cell or on an infectious agent. In further embodiments, the Fab domain is specific for HER2/neu, EGFR, or CD20. In another aspect, the present disclosure provides compositions and methods for treating cancer or an infectious disease in a subject, comprising administering a multimerizing stradobody to the subject, wherein the multimerizing stradobody comprises an Fab domain specific for an antigen expressed on a tumor or a cancer cell or on an infectious agent, and wherein the multimerizing stradobody is administered at a dose level of less than about 1 mg/kg. In further embodiments, the Fab domain is specific for HER2/neu, EGFR, or CD20.

### BRIEF DESCRIPTION OF THE FIGURES

FIG. 1 shows the binding of G001 (negative control), GB4500, or GB4542 at doses ranging from 0 μg/ml to 10 μg/ml on human peripheral blood B cells or on T cells, NK cells, monocytes, or granulocytes in the presence (+ rows) or absence (− rows) of B cells.

FIG. 2A is a graph showing the ADCC activity (presented as percent cytotoxicity) of GB4542, GB4500, or IVIG (negative control) in vitro over concentrations ranging from 0.001 to 100 μg/mL. FIG. 2B shows the ADCP activity (presented as percent phagocytosis) of GB4542, GB4500, or IVIG control in vitro over concentrations ranging from 0.000128 to 50 μg/mL. FIG. 2C shows the CDC activity (presented as percent cytotoxicity) GB4542, GB4500, or control IVIG in vitro over concentrations ranging from 0.00064 to 50 μg/mL.

FIG. 3A shows the binding of GB4542 (square symbols) or GB4500 (triangle symbols) to C1q. FIG. 3B shows the inhibition of GB4500-mediated B cell CDC by GB2542 (a stradobody having an Fab specific for Her2/neu antigen), its parent monoclonal antibody GB2500, or control IVIG. FIG. 3C shows the inhibition of GB4500-mediated ADCC by GB2542, GB2500, or control IVIG. FIG. 3D shows the inhibition of GB4500-mediated ADCP by GB2542, GB2500, or control IVIG.

FIG. 4A shows the percent B cell depletion in PBMC mediated by GB4542, GB4500, or IVIG over concentrations ranging from 0.001 to 100 μg/mL. FIG. 4B shows the induction of IL-10 (first panel from left), IL-12 (second panel from left), TNFα (third panel from left) or IL-6 (fourth panel from left) in PBMC by GB4542, GB4500, or IVIG in the presence of LPS.

FIG. 5 shows complement dependent B cell depletion in PBL (FIG. 5 left panel) or spleen (FIG. 5, right panel) cells from cynomolgus monkeys in response to GB4500, GB4542, or IVIG control over a range of concentrations.

FIG. 6 shows the depletion of B cells, as measured by the number of CD3-CD19+ cells per μL blood (left panels) or by the CD20+ mean fluorescence intensity (MFI; right panels), in cynomolgus monkeys over time following administration of GB4542 at a single dose level of 0.1 mg/kg (top panels) or 1 mg/kg (bottom panels).

FIG. 7 provides the number of lymphocytes and monocytes per μL blood for the 0.1 mg/kg dose (top three panels) and 1.0 mg/kg dose (bottom three panels) over time following GB4542 administration to cynomolgus monkeys. The two left panels are bar graphs showing the lymphocyte numbers per μL blood during infusion and on the indicated days post infusion (up to day 55 for the 0.1 mg/kg dose and up to day 103 for the 1.0 mg/kg dose) as measured by FACS. The line graphs in the middle and right side panels show the lymphocyte numbers (middle panels) and monocyte numbers (right panels) per μL blood during infusion and at the indicated time points up to 1000 hours post infusion, as measured by CBC test.

FIG. 8 shows the depletion of B cells in the peripheral blood of cynomolgus monkeys following administration of rituximab (left panel), obinutuzumab (left panel), or GB4542 (right panel). The left panel of FIG. 8 shows that rituximab (Rituxan; 2 doses of 10 mg/kg) or obinutuzumab (GA101; 2 doses of 10 mg/kg or 30 mg/kg) both deplete B cells in the peripheral blood of cynomolgus monkeys (n=3 per group). Mossner et al. Blood (2010) 115(22)4393-4402. The right panel of FIG. 8 shows the depletion of B cells in the peripheral blood of cynomolgus monkeys following repeated doses of GB4542 at a dose of 1 mg/kg every three days for 3 total doses. The data both panels are presented as the ratio of B cells to T cells in the peripheral blood at the indicated timepoints.

FIG. 9 shows the competitive NK cell binding with GO45c for a range of doses of GB4542. Cynomolgus monkeys were administered 0.1, 0.5, 1, or 10 mg/kg GB4542 by subcutaneous injection at day 0. Blood was drawn on day 1, 4, 7, and 14 and G045c binding to NK cells was assessed by flow cytometry. NK cells were identified as CD3-CD20− CD159a+ cells within the lymphocyte gate.

FIGS. 10A, 10B, 10C, and 10D show the activities of different molecular weight fractions of GB4542. FIG. 10A is a picture of a Coomassie gel showing three different fractions (FR1, FR2, and FR3) of GB4542 Stb-GGGSGH in lanes 2, 3, and 4, respectively. Lane 1 shows GB4500.

FIG. 10B is a set of line graphs showing the ADCP (left panel) and CDC (right panel) activities of increasing concentrations of FR2 and FR4 fractions of GB4542. *p<0.05, **p<0.01 FR2 vs FR4.

FIG. 10C is a set of bar graphs showing the total B cell number per μL of blood (top row of bar graphs) and the % depletion of B cells in the blood (bottom row of bar graphs) in monkeys on days 0, 1, 3, 7, and 14 following subcutaneous administration of GB4542 FR1 (left panels), GB4542 FR2 (middle panels), or GB4542 FR3 (right panels).

FIG. 10D is a line graph showing the % B cell depletion in the blood of monkeys on days 0, 1, 3, 7, and 14 following administration of GB4542 FR1, FR2, or FR3.

FIG. 11 shows an in silico analysis of the isoleucine zipper multimerization domain (SEQ ID NO: 99, contained in SEQ ID NO: 110).

FIG. 12 is a picture of a Coomassie gel showing the multimerization of GB4542 transient (having the unmodified multimerization domain) and multimerization of stable GB4542 stradobodies having the modified multimerization domains.

### DETAILED DESCRIPTION OF THE INVENTION

There is a need in the art to overcome the disadvantages of antibody therapy. The present inventors have developed a compound capable of combining the activity of target killing of an antigen-specific monoclonal antibody with immune tolerance induction that is intravenous immunoglobulin (IVIG)-like. IVIG has been used since the early 1950's to treat immune deficiency disorders and more recently, and more commonly, for autoimmune and inflammatory diseases. IVIG mediates immune tolerogenic effects via several mechanisms, including IVIG aggregate binding and cross-linking of Complement C1q and Fc gamma receptors (FcγRs) on immune cells including NK cells (e.g. FcγRIIIa), macrophages (e.g. FcγRIIa), B cells (e.g. FcγRIIb), and monocytes and monocyte derived cells including dendritic cells.

The present inventors have surprisingly found that while low doses of stradobodies and lower order multimer fractions of stradobodies mediate potent target cell depletion through mechanisms including, but not limited to, CDC, ADCC, and ADCP, high doses of stradobodies and higher order multimer fractions of stradobodies protect against inflammation and FcγR and complement-mediated cytotoxicity. Moreover, stradobodies exhibit potent inflammatory cytokine release only up to a threshold dose level required for target cell depletion, whereas at higher doses, pro-inflammatory cytokine induction is rapidly reduced. Thus, unlike traditional antibody molecules, which mediate dose-dependent increases in cell killing, pro-inflammatory cytokine induction, etc. even after optimal target cell depletion is achieved, stradobodies are an effective means to induce initial depletion, such as B cell depletion, and secondary “IVIG-like” tolerance. The differing effects—target cell depletion at low concentration or low dose or more limited drug exposure or lower ratio of higher order multimers and suppression of the immune response at higher concentration or higher dose or more sustained drug exposure or higher ratio of higher order multimers—we call “bimodal activity.” Surprisingly, when higher doses of the compounds disclosed herein are used initially, the target cells (such as B cells or tumor cells) are protected from depletion. The stradobodies provided herein therefore have the dual advantage of both antibody-mediated effector functions and IVIG-like immune suppressive or tolerogenic properties. Without being limited by theory, at low doses and lower order multimers including the homodimer the stradobody preferentially binds to the target via the Fab, leading to killing and/or depletion of the target bearing the antigen through a) subsequent complement deposition and Complement Dependent Cytotoxicity (“CDC”); b) Direct Cytotoxicity (“DC”); and c) immune cell binding to the Fc, inducing Antibody Dependent Cell Cytotoxicity (“ADCC”), Antibody Dependent Cellular phagocytosis (“ADCP”), and other such natural effector cell mechanisms. In yet another aspect, at higher doses and higher order multimers the binding of multivalent Fc of the stradobodies provided herein to immune cells outcompetes the binding of the multivalent Fab′ of the stradobodies provided herein to the target cell antigen. Without being limited by theory, this could occur in the absence of the antigenic target or because of saturation or internalization of the antigenic sites on the target cell. Therefore, at higher doses the stradobody preferentially binds to the immune cells and complement through its Fc domain and induces immunosuppression similar to that induced by IVIG, particularly immune suppression similar to the Fc portion of the aggregate fraction of IVIG. The mechanisms by which IVIG and bimodal stradobodies induce immunosuppression is diverse, multi-faceted, and varies by disease and involves changes in cell maturation and cell surface markers, release of pro-inflammatory cytokines followed by anti-inflammatory cytokines, binding to complement factors, and numerous other published observations.

In one embodiment, the target cell depleting properties of the stradobodies disclosed herein occur when the antigen target is in excess of the stradobody, such that the stradobody Fab preferentially binds with avidity because of the two Fab′ domains to the target antigen to a greater degree than the stradobody Fc binds to Fc receptors. In one embodiment, the IVIG-like immune suppressive or tolerogenic properties of the stradobodies disclosed herein occur when the stradobody is in excess of the antigen target, such that the Fc domain of the stradobody preferentially binds to complement and to FcγRs on immune cells such as monocytes, macrophages, dendritic cells, B cells, and NK cells to a greater degree than the stradobody Fab binds to the target antigen. The resulting binding of complement and the FcγRs on immune cells, in one embodiment, induces immunologic tolerance.

In one embodiment, the target cell depleting properties of the stradobodies disclosed herein occur in particular when the stradobody is a homodimer or a lower order multimer such as the dimer of the homodimer or weighted in its composition towards the homodimer and the dimer. In another embodiment, the IVIG-like immune suppressive or tolerogenic properties of the stradobodies disclosed herein occur when the stradobody is a higher order multimer. A stradobody that is a “higher order multimer” refers to a stradobody comprising multimers of the homodimer, or weighted in its compositions towards the higher order multimers of the homodimer, in particular presenting 3, 4, 5, 6, 7, 8, 9, 10, 11, 12 or more Fc simultaneously to low affinity Fc receptors and to complement. In some embodiments, the higher order multimers may be tetramers, pentamers, hexamers, heptamers, octamers, nonamers, or decamers.

In one aspect, the present disclosure provides methods for inducing target cell depletion followed by suppression of inflammation in a subject, the method comprising (i) administering a first multimerizing stradobody that is predominantly a homodimer or a lower order multimer, wherein the first multimerizing stradobody results in target cell depletion; and (ii) administering a second multimerizing stradobody, wherein the second multimerizing stradobody is predominantly a higher order multimer, and wherein the second multimerizing stradobody results in suppression of inflammation in the subject. In some embodiments, the first and second stradobody have the same structure and/or the same antibody specificity, and differ only in that the first stradobody is present predominantly as a homodimer or a lower order multimer and the second stradobody is present predominantly as a higher order multimer. “Predominantly” is used interchangeably with “primarily” herein. In some embodiments, stradobodies that are predominantly a homodimer or a lower order multimer are comprised of more than about more than about 50%, more than about 60%, more than about 70%, more than about 80%, or more multimer bands that are lower order multimers (e.g., the homodimer and the dimer of the homodimer). Stradobodies that are predominantly higher order multimers are comprised of more than about 50%, more than about 60%, more than about 70%, more than about 80%, or more multimer bands at higher orders than the homodimer and dimer of the homodimer. In some embodiments, the first and second stradobody have the identical amino acid sequence, and differ only in that the first stradobody is primarily present as a homodimer or a lower order multimer and the second stradobody is primarily present as a higher order multimer.

In some embodiments, the ratio of higher order versus lower order multimer bands on gel analysis is controlled such that an optimal ratio of bands is present for the bimodal activity of the stradobodies. In some embodiments, the ratio of higher order to lower order bands is controlled using chromatographic separation, such as, for example, size exclusion chromatography, ion exchange chromatography (e.g., anion or cation exchange chromatography), or hydrophobic interaction chromatography. For example, in some embodiments, the largest 1%, 5%, 10%, 20%, or more of the compound is separated out, in order to enrich for the mAb-like effect of the lower order multimers over the otherwise unfractionated Protein A purified protein. In other embodiments, the smallest 1%, 5%, 10%, or 20% or more of the compound is separated out, in order to enrich for the IVIG-like effect of the higher-order multimers over the otherwise unfractionated Protein A purified protein. In other embodiments, the ratio of higher order to lower order bands is controlled by adding the mAb or a stradomer to the compound mix. For example, in some embodiments, a monoclonal antibody (e.g., a monoclonal antibody having the same antigen specificity and/or the identical Fab as the stradobody) is added at 1%, 5%, 10%, 20%, or more to the unfractionated or fractionated stradobody. In such embodiments, a mix of, for example, 20% mAb and 80% stradobody is generated to obtain increased target effect. As another example, a stradomer (e.g., a stradomer having the same antigen specificity and/or the identical Fab as the stradobody) is added at 1%, 5%, 10%, 20%, or more to the unfractionated or fractionated stradobody. In such embodiments, a mix of, for example, 20% stradomer and 80% stradobody is generated to obtain increased tolerance.

In one aspect, the IVIG-like immune suppression of the higher order multimers occurs by increased binding of hexameric C1q. In another aspect, the IVIG-like immune suppression of the higher order multimers occurs by presentation of polyvalent Fc to Fc receptors. In still another aspect, the stradobody activity of the higher order multimers is directed via the Fab to the antigen of interest where site-directed tolerance occurs. Without being limited by theory, the site-directed tolerance can occur by the mechanisms of binding of complement and engagement of low affinity Fc receptors by the polyvalent Fc of the stradobody higher order multimers. Thus, in one embodiment, a stradobody having the same structure and antigen specificity exhibits target depletion activity when present as a homodimer or a lower order multimer; and exhibits immune suppression that may be site directed when present as a higher order multimer.

In one aspect, the higher order multimers of the stradobodies provided herein bind to immune cells and to unbound complement. For example, because the stradobody is multimeric, unlike a monoclonal antibody that must be bound to a cell in close proximity to other monoclonal antibodies in order to bind C1q effectively, the stradobody comprising the same or similar Fab can effectively bind C1q without being cell-bound, resulting in inhibition of CDC as a means of achieving tolerance. Thus, the higher order multimers of stradobodies bind preferentially to complement relative to a mAb with the identical Fab, which will first bind its target antigen through its Fab with avidity and only then bind to C1q (inducing CDC) and immune cells such as NK cells (ADCC) and macrophages (ADCP) through its Fc domain. For example, in some embodiments, a stradobody (e.g., 4542) having an Fc:Fab ratio of 2, such that the third multimer band presents 6 Fc and 3 Fab, avidly binds the C1q hexamer, thus acting as a complement sink in the blood away from the target tissue and preventing C1q from binding after the Fab binding to the target, thus preventing CDC.

In one embodiment, the stradobody is more potent than an equimolar amount of the mAb sharing the identical Fab. For example, in one embodiment, the stradobody exhibits higher killing of cells expressing the target antigen recognized by the Fab, relative to the mAb sharing the identical Fab. As another example, in one embodiment, the stradobody is more immune suppressive or tolerogenic relative to the mAb sharing the identical Fab.

The stradobodies provided in the present disclosure are immunologically active biomimetic(s) comprising an Fab domain and an Fc domain. For example, in one embodiment, the stradobodies provided in the present disclosure comprise an Fab domain and a stradomer. Stradomers are disclosed herein and have been described, for example, in U.S. Patent Application Publication Nos. US 2010-0239633 and US 2013-0156765, incorporated by reference herein in their entireties. In a further embodiment, the first dose level of the stradobody is a low dose of stradobody. The biomimetics exhibit a bimodal dose-response profile such that in the presence of antigen, at low concentrations or doses of stradobody, the biomimetics exhibit strong Ab-mediated effector functions, while at high concentrations or doses, the biomimetics exhibit Fc-mediated tolerance. Thus, the biomimetics provided herein offer the advantage of specific cell depletion as well as immunological tolerance. For example, in one embodiment, the biomimetics may be administered in such a manner that specific cell depletion is followed by immunological tolerance. In another embodiment, the biomimetics may be administered in such a manner that opsonization and destruction of an infectious agent is followed by immunological tolerance. The compounds have utility for treating, for example, autoimmune and inflammatory diseases and cancers, as well as infectious diseases.

In one embodiment, the stradobodies comprise an Fab domain that is specific for a target antigen. In one embodiment, the target antigen is present on a cell or on an infectious agent (e.g Rho(D), EGFR, Her2/neu). In another embodiment, the target antigen is a soluble antigen such as a cytokine (e.g., TNFα, IL17, and IL12/23).

Target cell depletion and target infectious agent destruction may be via any Fc function or combination of Fc functions. Fc functions include cytotoxicity including antibody-dependent cell-mediated cytotoxicity (ADCC), complement-dependent cell cytotoxicity (CDC), direct cell cytotoxicity, and other mechanisms of cellular toxicity, as well as antibody-dependent cellular phagocytosis (ADCP). In some embodiments, the Fc functions are 5, 10, 50, 100, 500, 1000, or more times more potent relative to a monoclonal antibody comprising the identical Fab against the same antigen. ADCC is a mechanism by which NK cells kill other cells. For example, the Fc portions of antibodies bound to a target cell interact with Fc receptors that are expressed by effector cells, thereby initiating signaling cascades that result in the release of cytotoxic granules, which induce apoptosis of the antibody-targeted cell. CDC is a mechanism of killing cells in which an antibody, bound to the target cell surface, fixes complement, which results in assembly of the membrane attack complex that punches holes in the target cell membrane resulting in subsequent cell lysis. ADCP is a mechanism of phagocytosis wherein binding of Fc receptors on phagocytes to multivalent antibody-coated particles leads to engulfment of the particles and the activation of phagocytes. The particles are internalized into vesicles known as phagosomes, which fuse with lysosomes, and the phagocytosed particles are destroyed in these phagolysosomes. As an example, a stradobody having an Fab domain that is specific for an antigen on an infectious agent (e.g., a virus) may bind the virus and opsonize it for destruction.

In some embodiments, a low in vitro concentration of stradobodies is a concentration of less than 100 μg/mL less than 80 μg/mL, less than 60 μg/mL, less than 50 μg/mL, less than 40 μg/mL, less than 30 μg/mL, less than 20 μg/mL, less than 10 μg/mL, less than 9 μg/mL, less than 8 μg/mL, less than 7 μg/mL, less than 6 μg/mL, less than 5 μg/mL, less than 4 μg/mL, less than 3 μg/mL, less than 2 μg/mL, less than 1 μg/mL, less than 0.9 μg/mL, less than 0.8 μg/mL, less than 0.7 μg/mL, less than 0.6 μg/mL, less than 0.5 μg/mL, less than 0.4 μg/mL, less than 0.3 μg/mL, less than 0.2 μg/mL, less than 0.1 μg/mL, less than 0.05 μg/mL, or less than 0.01 μg/mL. In other embodiments, a low in vivo dose is less than 10 mg/kg, less than 9 mg/kg, less than 8 mg/kg, less than 7 mg/kg, less than 6 mg/kg, less than 5 mg/kg, less than 4 mg/kg, less than 3 mg/kg, less than 2 mg/kg, less than 1 mg/kg, less than 0.5 mg/kg, less than 0.1 mg/kg, less than 0.05 mg/Kg, or less than 0.01 mg/Kg.

In some embodiments, a high in vitro concentration of stradobodies is a concentration of more than 10 μg/mL, more than 50 μg/mL, more than 75 μg/mL, more than 100 μg/mL, more than 250 μg/mL, more than 500 μg/mL, or more than 1000 μg/mL. In other embodiments, a high in vivo dose is more than 0.5 mg/Kg, more than 1 mg/kg, more than 5 mg/kg, more than 10 mg/kg, more than 25 mg/kg, more than 50 mg/kg, more than 100 mg/kg, or more than 500 mg/kg.

In some embodiments, the mg/kg in vivo dosing level is determined by calculating the estimated circulating blood volume of the subject or group of subjects to receive the stradobody.

In one embodiment, a stradobody may be used clinically primarily for cell killing. As an example, in treating most cancers, such as a HER2/neu breast cancer or an EGFR-expressing colon cancer, it is clinically desirable to kill tumor cells rather than to induce tolerance. In such embodiments the stradobody will be used at low doses and with high ratios of homodimer and dimer to maximize cell killing. Similarly, in treating certain infectious diseases, for example E. coli-mediated colitis, Staphylococcus abscess, or pulmonary tuberculosis, it is clinically desirable to kill the organism rather than to induce tolerance to the organism. In such embodiments the stradobody will be used at low doses and/or with high ratios of homodimer and dimer to maximize killing of the infectious agent.

In another embodiment, a stradobody may be used clinically both for cell killing and for induction of tolerance. For example, in some embodiments, a stradobody against proinflammatory targets (e.g. TNF, IL17, IL12/23) will be used in this manner. Similarly, much of the morbidity and mortality of certain infectious diseases is from the body's inflammatory reaction to the infection. Examples of such infectious diseases include Ebola disease in which the body's inflammatory response to the virus is potentially fatal; Aspergellosis in which airway inflammation is caused by the presence of the fungus; and the acneiform reaction and inflammatory response leading to prostate cancer associated with P. acne colonization. In some embodiments, the present disclosure provides methods for treatment of such inflammatory and infectious diseases comprising administering a low dose and/or low multimer bands followed by a high dose and/or higher order multimer bands. In other embodiments, the methods comprise administering the monoclonal antibody targeting the same antigen followed by a high dose and/or higher order multimer bands of the stradobody. As a further embodiment, the methods comprise inducing tolerance to such inflammation and inflammatory response to infectious agents with treatment of the stradobody alone. In one embodiment, such tolerance induction is accomplished with just a high dose of stradobody and/or with a stradobody selected to have a high ratio of higher order multimers compared with the homodimer and dimer.

Stradobody Structure

In some embodiments, the biomimetics of the present invention have at least two Fc domains, and at least one Fab domain. In some embodiments, the Fc domain portion of the biomimetic is a stradomer. Stradomers are biomimetic compounds capable of binding two or more Fc receptors, preferably two or more Fcγ receptors, and more preferably demonstrating significantly improved binding relative to an Fc domain and most preferably demonstrating slow dissociation characteristic of avidity. The physical stradomer conformations have been previously described in U.S. Patent Application Publication No. 2010/0239633, and PCT Publication No. WO 2012/016073, both of which are incorporated by reference herein in their entireties. An exemplary stradomer is G045c. G045c has the structure: IgG1 Hinge-IgG1CH2 IgG1 CH3-IgG2 Hinge.

The biomimetics provided herein comprising at least two Fc domains and at least one Fab domain are termed “stradobodies.” As used herein, “stradobody” refers to a molecule comprising two or more Fc domains, to which one or more Fab domain is attached. Thus, by virtue of such Fab domains and Fc domains, stradobodies have both antigen binding capacity and Fcγ receptor binding activity. In some embodiments, the Fcγ receptor activity may be due to an ability to bind and cross-link FcγR equal to or greater than the Fc portion of a native structure holo-antibody. For example, in some embodiments, the biomimetic compounds provided herein comprise a stradobody, which comprises at least one Fc domain, and an Fab domain. Stradobodies have been previously described in U.S. Patent Application Publication Nos. US 2010-0239633, US 2013-0156765, and US-2014-0072582, each of which is incorporated herein by reference in its entirety for all purposes. In some embodiments, the stradobodies are multimerizing stradobodies.

As used herein, “Fc domain” describes the minimum region (in the context of a larger polypeptide) or smallest protein folded structure (in the context of an isolated protein) that can bind to or be bound by one or more Fc γ-receptor (FcγR) (e.g., FcγRI, FcγRIIa, FcγRIIb, FcγRIIIa, FcγRIIIb and FcγRIV); FcRn; DC-SIGN; SIGN-R1; TRIM21; Dectin-1; Fc Receptor Like Molecules FCRL1-6, FCRLA, and FCRLB; complement components C1q, C3, C3a, C3b, C4, or C4a. In both an Fc fragment and an Fc partial fragment, the Fc domain is the minimum binding region that allows binding of the molecule to an Fc receptor. While an Fc domain can be limited to a discrete homodimeric polypeptide that is bound by an Fc receptor, it will also be clear that an Fc domain can be a part or all of an Fc fragment, as well as part or all of an Fc partial fragment. When the term “Fc domains” is used in this invention it will be recognized by a skilled artisan as meaning more than one Fc domain. An Fc domain is comprised of two Fc domain monomers. As further defined herein, when two such Fc domain monomers associate, the resulting Fc domain has Fc receptor binding activity. Thus an Fc domain is a dimeric structure that can bind an Fc receptor.

At a minimum, an Fc domain is a dimeric polypeptide (or a dimeric region of a larger polypeptide) that comprises two peptide chains or arms (monomers) that associate to form a functional Fcγ receptor binding site. Therefore, the functional form of the individual Fc fragments and Fc domains discussed herein generally exist in a dimeric (or multimeric) form. Further, the Fc fragments and Fc domains generally exist in homodimeric form. The monomers of the individual fragments and domains discussed herein are the single chains or arms that must associate with a second chain or arm to form a functional dimeric structure.

As used herein, “Fc domain monomer” describes the single chain protein that, when associated with another Fc domain monomer, comprises an Fc domain that can bind to an Fcγ receptor. The association of two Fc domain monomers creates one Fc domain. An Fc domain monomer alone, comprising only one side of an Fc domain, cannot bind an Fcγ receptor. As used herein, “Fc partial domain monomer” describes the single chain protein that, when associated with another Fc partial domain monomer, comprises an Fc partial domain. The association of two Fc partial domain monomers creates one Fc partial domain.

The term “Fab domain” describes the minimum region (in the context of a larger polypeptide) or smallest protein folded structure (in the context of an isolated protein) that can bind to an antigen. The Fab domain is the minimum binding region of an Fab fragment that allows binding of the molecule to an antigen. “Fab domain” is used interchangeably herein with “Fab”. The Fab portion of the stradobody may comprise both a heavy and a light chain. The variable heavy chain and the light chain may be independently from any compatible immunoglobulin such as IgA1, IgA2, IgM, IgD, IgE, IgG1, IgG2, IgG3, or IgG4, and may be from the same or different Ig isotype, but preferably are from the same Ig isotype. The light chains kappa or lambda may also be from different Ig isotypes. In some embodiments, stradobodies, like stradomers, can bind two or more FcγRs and modulate immune function. In one embodiment, the stradobodies of the current invention comprise a Fab domain, one or more Fc domains, and one or more multimerization domains, wherein at least one of the one or more multimerization domains separates two or more Fc domains, or is located at the carboxy end of the Fc region.

Through the Fab domain, the immunologically active biomimetics of the present invention are capable of binding to one or more antigens. In some embodiments, the immunologically active biomimetics of the present invention are capable of binding to two different antigens, similar to bispecific antibodies. In other embodiments, the immunologically active biomimetics of the present invention are capable of binding to more than two different antigens. The biomimetics of the present invention also possess one or more immune modulating activities of the IgG Fc domain and have at least a first Fc domain capable of binding one or more Fc γ-receptor (FcγR) (e.g., FcγRI, FcγRIIa, FcγRIIb, FcγRIIIa, FcγRIIIb and FcγRIV); FcRn; DC-SIGN; SIGN-R1; TRIM21; Dectin-1; Fc Receptor Like Molecules FCRL1-6, FCRLA, and FCRLB; or complement components C1q, C3, C3a, C3b, C4, or C4a. In some embodiments, the biomimetics of the present invention possess a second Fc domain capable of binding one or more Fc γ-receptor (FcγR) (e.g., FcγRI, FcγRIIa, FcγRIIb, FcγRIIIa, FcγRIIIb and FcγRIV); FcRn; DC-SIGN; SIGN-R1; TRIM21; Dectin-1; Fc Receptor Like Molecules FCRL1-6, FCRLA, and FCRLB; or complement components C1q, C3, C3a, C3b, C4, or C4a. Thus, when multimerized, the immunologically active biomimetics contain at least two dimeric structures, each possessing the ability to bind to one or more antigens, and the ability to bind to one or more Fc γ-receptor (FcγR) (e.g., FcγRI, FcγRIIa, FcγRIIb, FcγRIIIa, FcγRIIIb and FcγRIV); FcRn; DC-SIGN; SIGN-R1; TRIM21; Dectin-1; Fc Receptor Like Molecules FCRL1-6, FCRLA, and FCRLB; or complement components C1q, C3, C3a, C3b, C4, or C4a.

The term “Fc region” is used herein to refer to the region of the stradobody that comprises Fc domains, domain linkages, and multimerization domains. Thus, the Fc region is the region of the stradobody that does not comprise the Fab domain.

Multimerization domains are described, for example, in U.S. Patent Application Publication Nos. US 2013-0156765 and US 2014-0072582, incorporated by reference in their entireties for all purposes. Multimerization domains are amino acid sequences known to cause protein multimerization in the proteins where they naturally occur. The multimerization domain may comprise a peptide sequence that causes dimeric proteins to further multimerize. “Multimerization,” as used herein, refers to the linking or binding together of multiple (i.e., two or more) individual stradobody homodimers. For example, stradobodies are multimerized when at least one stradobody homodimer (i.e., at least one homodimeric polypeptide comprising one or more Fc domains and one or more Fab domains) is attached to at least one other stradobody homodimer via a multimerization domain. Examples of peptide multimerization domains include IgG2 hinge, isoleucine zipper, collagen Glycine-Proline-Proline repeat (“GPP”) and zinc fingers. In one embodiment, the multimerization domains may be IgG hinges, isoleucine zippers, or a combination thereof. In one embodiment, the stradobody is comprised of an Fab, one or more Fc domain, and one or more multimerization domain independently selected from the group consisting of an IgG2 hinge, an isoleucine zipper, and a GPP repeat. In a particular embodiment, the stradobody is comprised of an Fab, a first Fc domain, an isoleucine zipper, an IgG2 hinge, and a second Fc domain.

In one embodiment, stradobody comprises a sequence according to SEQ ID NO: 32, which includes an unmodified isoleucine zipper and a restriction site. In other embodiments, the isoleucine zipper comprises a sequence according to SEQ ID NO: 99. In other embodiments, the isoleucine zipper comprises a modified amino acid sequence. For example, in some embodiments, the isoleucine zipper comprises a sequence according to SEQ ID NO: 100 (GGGS removed from the amino terminus of the isoleucine zipper), 101 (GH removed from the carboxy terminus of the isoleucine zipper), or 102 (GGGS at amino terminus and GH at carboxy terminus both removed from the isoleucine zipper). Exemplary stradobodies comprising modified isoleucine zippers are GB4542 Stable-GGGS (SEQ ID NO: 96), GB4542 Stable-GH (SEQ ID NO: 97), and GB4542 Stb-GGGSGH (SEQ ID NO: 99). However, any stradobody comprising an isoleucine zipper according to any one of SEQ ID NOs: 32, 99, 100, 101, or 102 are included in the disclosure, such as, for example, stradobodies comprising an Fab′ specific for HER2/neu, EGFR, TNF, Rho(D), IL17, and IL12/23.

As indicated above, each of Fc fragments, Fc partial fragments, Fc domains and Fc partial domains are dimeric proteins or domains. Thus, each of these molecules is comprised of two monomers that associate to form the dimeric protein or domain.

Exemplary Stradobodies

The stradobodies provided herein may comprise any Fab region. Exemplary stradobodies and the corresponding monoclonal antibodies having the same Fab region are provided in the table below. The stradobodies disclosed herein and provided below have been described, for example, in U.S. Patent Application Publication No. US-2014-0072582.

TABLE 1
Unaltered monoclonal antibodies and exemplary stradobody compounds

| | |
|---|---|
| Compound | Specificity |

Monoclonal antibodies

| | |
|---|---|
| GB2500 | HER2/neu |
| (trastuzumab) | |
| GB3500 | EGFR |
| (cetuximab) | |
| GB4500 | CD20 |
| (rituximab) | |
| GB7500 | TNF |
| (adalimumab) | |
| GB9500 | Rho(D) |
| GB10500 | IL-17 |
| (secukinumab) | |
| GB11500 | IL12/23 |
| (ustekinumab) | |

Multimerizing serial stradobodies

| | |
|---|---|
| GB2524 | HER2/neu |
| GB2538 | HER2/neu |
| GB2540 | HER2/neu |
| GB2542 | HER2/neu |
| GB3524 | EGFR |
| GB3538 | EGFR |
| GB3540 | EGFR |
| GB3542 | EGFR |
| GB4524 | CD20 |
| GB4538 | CD20 |
| GB4540 | CD20 |
| GB4542 | CD20 |
| GB7524 | TNF |
| GB7538 | TNF |
| GB7540 | TNF |
| GB7542 | TNF |

Non-multimerizing serial stradobodies

| | |
|---|---|
| GB2554 | HER2/neu |
| GB2555 | HER2/neu |
| GB3554 | EGFR |
| GB3555 | EGFR |
| GB4554 | CD20 |
| GB4555 | CD20 |
| GB7554 | TNF |
| GB7555 | TNF |

C-terminal multimerized stradobodies

| | |
|---|---|
| GB2534 | HER2/neu |
| GB2545 | HER2/neu |
| GB2546 | HER2/neu |
| GB2547 | HER2/neu |
| GB2549 | HER2/neu |
| GB2550 | HER2/neu |
| GB2560 | HER2/neu |
| GB2561 | HER2/neu |
| GB2562 | HER2/neu |
| GB2563 | HER2/neu |
| GB2589 | HER2/neu |
| GB2590 | HER2/neu |
| GB3534 | EGFR |
| GB3545 | EGFR |
| GB3546 | EGFR |
| GB3547 | EGFR |
| GB3549 | EGFR |
| GB3550 | EGFR |
| GB3560 | EGFR |
| GB3561 | EGFR |
| GB3562 | EGFR |
| GB3563 | EGFR |
| GB3589 | EGFR |
| GB3590 | EGFR |
| GB4534 | CD20 |
| GB4545 | CD20 |
| GB4546 | CD20 |
| GB4547 | CD20 |
| GB4549 | CD20 |
| GB4550 | CD20 |
| GB4560 | CD20 |
| GB4561 | CD20 |
| GB4562 | CD20 |
| GB4563 | CD20 |
| GB4589 | CD20 |
| GB4590 | CD20 |
| GB7534 | TNF |
| GB7545 | TNF |
| GB7546 | TNF |
| GB7547 | TNF |
| GB7549 | TNF |
| GB7550 | TNF |
| GB7560 | TNF |
| GB7561 | TNF |
| GB7562 | TNF |
| GB7563 | TNF |
| GB7589 | TNF |
| GB7590 | TNF |
| GB9545 | Rho(D) |
| GB9542 | Rho(D) |
| GB10542 | IL17 |
| GB10545 | IL17 |
| GB11542 | IL12/23 |
| GB11545 | IL12/23 |

TABLE 2
Amino acid sequences of exemplary stradobody compounds and components of the
stradobody compounds.

| | Sequence |
|---|---|
| Leader sequence | METDTLLLWVLLLWVPGSTG |
| (SEQ ID NO: 1) | |
| GB2542 Variable and | EVQLVESGGGLVQPGGSLRLSCAASGFNIKDTYIHWVRQA |
| CH1 regions (identical | PGKGLEWVARIYPTNGYTRYADSVKGRFTISADTSKNTAY |
| to variable and CH1 | LQMNSLRAEDTAVYYCSRWGGDGFYAMDYWGQGTLVT |
| regions of | VSSASTKGPSVFPLAPSSKSTSGGTAALGCLVKDYFPEPVT |
| trastuzumab/GB2500) | VSWNSGALTSGVHTFPAVLQSSGLYSLSSVVTVPSSSLGTQ |
| (SEQ ID NO: 34) | TYICNVNHKPSNTKVDKKV |
| GB3542 Variable and | QVQLKQSGPGLVQPSQSLSITCTVSGFSLTNYGVHWVRQS |
| CH1 regions (identical | PGKGLEWLGVIWSGGNTDYNTPFTSRLSINKDNSKSQVFF |
| to variable and CH1 | KMNSLQSNDTAIYYCARALTYYDYEFAYWGQGTLVTVSA |
| regions of | ASTKGPSVFPLAPSSKSTSGGTAALGCLVKDYFPEPVTVS |
| cetuximab/GB3500) | WNSGALTSGVHTFPAVLQSSGLYSLSSVVTVPSSSLGTQT |
| (SEQ ID NO: 31) | YICNVNHKPSNTKVDKRV |
| GB4542 Variable and | QVQLQQPGAELVKPGASVKMSCKASGYTFTSYNMHWVK |
| CH1 regions (identical | QTPGRGLEWIGAIYPGNGDTSYNQKFKGKATLTADKSSST |
| to variable and CH1 | AYMQLSSLTSEDSAVYYCARSTYYGGDWYFNVWGAGTT |
| regions of | VTVSAASTKGPSVFPLAPSSKSTSGGTAALGCLVKDYFPEP |
| rituximab/GB4500) | VTVSWNSGALTSGVHTFPAVLQSSGLYSLSSVVTVPSSSLG |
| (SEQ ID NO: 36) | TQTYICNVNHKPSNTKVDKKV |
| GB7542 Variable and | EVQLVESGGGLVQPGRSLRLSCAASGFTFDDYAMHWVRQ |
| CH1 regions (identical | APGKGLEWVSAITWNSGHIDYADSVEGRFTISRDNAKNSL |
| to variable and CH1 | YLQMNSLRAEDTAVYYCAKVSYLSTASSLDYWGQGTLVT |
| regions of | VSSASTKGPSVFPLAPSSKSTSGGTAALGCLVKDYFPEPVT |
| adalimumab/GB7500) | VSWNSGALTSGVHTFPAVLQSSGLYSLSSVVTVPSSSLGTQ |
| (SEQ ID NO: 67) | TYICNVNHKPSNTKVDKKV |
| IgG1 Fc | EPKSCDKTHTCPPCPAPELLGGPSVFLFPPKPKDTLMISRTP |
| (SEQ ID NO: 2) | EVTCVVVDVSHEDPEVKFNWYVDGVEVHNAKTKPREEQ |
| | YNSTYRVVSVLTVLHQDWLNGKEYKCKVSNKALPAPIEK |
| | TISKAKGQPREPQVYTLPPSREEMTKNQVSLTCLVKGFYPS |
| | DIAVEWESNGQPENNYKTTPPVLDSDGSFFLYSKLTVDKS |
| | RWQQGNVFSCSVMHEALHNHYTQKSLSLSPGK |
| Isoleucine Zipper (ILZ) | GGGSIKQIEDKIEEILSKIYHIENEIARIKKLIGERGHDI |
| with restriction site (DI) | |
| (SEQ ID NO: 32) | |
| ILZ | GGGSIKQIEDKIEEILSKIYHIENEIARIKKLIGERGH |
| (SEQ ID NO: 99) | |
| Modified ILZ | IKQIEDKIEEILSKIYHIENEIARIKKLIGERGH |
| (SEQ ID NO: 100) | |
| Modified ILZ | GGGSIKQIEDKIEEILSKIYHIENEIARIKKLIGER |
| (SEQ ID NO: 101) | |
| Modified ILZ | IKQIEDKIEEILSKIYHIENEIARIKKLIGER |
| (SEQ ID NO: 102) | |
| IgG2 Hinge | ERKCCVECPPCP |
| (SEQ ID NO: 3) | |
| GB2542 Construct | METDTLLLWVLLLWVPGSTGEVQLVESGGGLVQPGGSLR |
| (SEQ ID NO: 35) | LSCAASGFNIKDTYIHWVRQAPGKGLEWVARIYPTNGYTR |
| | YADSVKGRFTISADTSKNTAYLQMNSLRAEDTAVYYCSR |
| | WGGDGFYAMDYWGQGTLVTVSSASTKGPSVFPLAPSSKS |
| | TSGGTAALGCLVKDYFPEPVTVSWNSGALTSGVHTFPAVL |
| | QSSGLYSLSSVVTVPSSSLGTQTYICNVNHKPSNTKVDKK |
| | VEPKSCDKTHTCPPCPAPELLGGPSVFLFPPKPKDTLMISRT |
| | PEVTCVVVDVSHEDPEVKFNWYVDGVEVHNAKTKPREE |
| | QYNSTYRVVSVLTVLHQDWLNGKEYKCKVSNKALPAPIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNHYTQKSLSLSPGKSLEGG |
| | GSIKQIEDKIEEILSKIYHIENEIARIKKLIGERGHDIERKCCV |
| | ECPPCPRLEGPRFEEPKSCDKTHTCPPCPAPELLGGPSVFLF |
| | PPKPKDTLMISRTPEVTCVVVDVSHEDPEVKFNWYVDGV |
| | EVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSD |
| | GSFFLYSKLTVDKSRWQQGNVFSCSVMHEALHNHYTQKS |
| | LSLSPGK |
| GB3542 Construct | METDTLLLWVLLLWVPGSTGQVQLKQSGPGLVQPSQSLSI |
| (SEQ ID NO: 33) | TCTVSGFSLTNYGVHWVRQSPGKGLEWLGVIWSGGNTDY |
| | NTPFTSRLSINKDNSKSQVFFKMNSLQSNDTAIYYCARALT |
| | YYDYEFAYWGQGTLVTVSAASTKGPSVFPLAPSSKSTSGG |
| | TAALGCLVKDYFPEPVTVSWNSGALTSGVHTFPAVLQSSG |
| | LYSLSSVVTVPSSSLGTQTYICNVNHKPSNTKVDKRVEPKS |
| | CDKTHTCPPCPAPELLGGPSVFLFPPKPKDTLMISRTPEVTC |
| | VVVDVSHEDPEVKFNWYVDGVEVHNAKTKPREEQYNST |
| | YRVVSVLTVLHQDWLNGKEYKCKVSNKALPAPIEKTISKA |
| | KGQPREPQVYTLPPSREEMTKNQVSLTCLVKGFYPSDIAV |
| | EWESNGQPENNYKTTPPVLDSDGSFFLYSKLTVDKSRWQ |
| | QGNVFSCSVMHEALHNHYTQKSLSLSPGKSLEGGGSIKQI |
| | EDKIEEILSKIYHIENEIARIKKLIGERGHDIERKCCVECPPCP |
| | RLEGPRFEEPKSCDKTHTCPPCPAPELLGGPSVFLFPPKPKD |
| | TLMISRTPEVTCVVVDVSHEDPEVKFNWYVDGVEVHNAK |
| | TKPREEQYNSTYRVVSVLTVLHQDWLNGKEYKCKVSNK |
| | ALPAPIEKTISKAKGQPREPQVYTLPPSREEMTKNQVSLTC |
| | LVKGFYPSDIAVEWESNGQPENNYKTTPPVLDSDGSFFLY |
| | SKLTVDKSRWQQGNVFSCSVMHEALHNHYTQKSLSLSPG |
| | K |
| GB4542 Construct | METDTLLLWVLLLWVPGSTGQVQLQQPGAELVKPGASVK |
| (SEQ ID NO: 37) | MSCKASGYTFTSYNMHWVKQTPGRGLEWIGAIYPGNGDT |
| | SYNQKFKGKATLTADKSSSTAYMQLSSLTSEDSAVYYCA |
| | RSTYYGGDWYFNVWGAGTTVTVSAASTKGPSVFPLAPSS |
| | KSTSGGTAALGCLVKDYFPEPVTVSWNSGALTSGVHTFPA |
| | VLQSSGLYSLSSVVTVPSSSLGTQTYICNVNHKPSNTKVDK |
| | KVEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPI |
| | EKTISKAKGQPREPQVYTLPPSREEMTKNQVSLTCLVKGF |
| | YPSDIAVEWESNGQPENNYKTTPPVLDSDGSFFLYSKLTV |
| | DKSRWQQGNVFSCSVMHEALHNHYTQKSLSLSPGKSLEG |
| | GGSIKQIEDKIEEILSKIYHIENEIARIKKLIGERGHDIERKCC |
| | VECPPCPRLEGPRFEEPKSCDKTHTCPPCPAPELLGGPSVFL |
| | FPPKPKDTLMISRTPEVTCVVVDVSHEDPEVKFNWYVDGV |
| | EVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSD |
| | GSFFLYSKLTVDKSRWQQGNVFSCSVMHEALHNHYTQKS |
| | LSLSPGK |
| GB7542 Construct | METDTLLLWVLLLWVPGSTGEVQLVESGGGLVQPGRSLR |
| (SEQ ID NO: 66) | LSCAASGFTFDDYAMHWVRQAPGKGLEWVSAITWNSGHI |
| | DYADSVEGRFTISRDNAKNSLYLQMNSLRAEDTAVYYCA |
| | KVSYLSTASSLDYWGQGTLVTVSSASTKGPSVFPLAPSSKS |
| | TSGGTAALGCLVKDYFPEPVTVSWNSGALTSGVHTFPAVL |
| | QSSGLYSLSSVVTVPSSSLGTQTYICNVNHKPSNTKVDKK |
| | VEPKSCDKTHTCPPCPAPELLGGPSVFLFPPKPKDTLMISRT |
| | PEVTCVVVDVSHEDPEVKFNWYVDGVEVHNAKTKPREE |
| | QYNSTYRVVSVLTVLHQDWLNGKEYKCKVSNKALPAPIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNHYTQKSLSLSPGKSLEGG |
| | GSIKQIEDKIEEILSKIYHIENEIARIKKLIGERGHDIERKCCV |
| | ECPPCPRLEGPRFEEPKSCDKTHTCPPCPAPELLGGPSVFLF |
| | PPKPKDTLMISRTPEVTCVVVDVSHEDPEVKFNWYVDGV |
| | EVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSD |
| | GSFFLYSKLTVDKSRWQQGNVFSCSVMHEALHNHYTQKS |
| | LSLSPGK |
| GB9542 | METDTLLLWVLLLWVPGSTGQVKLLESGGGVVQPGGSLR |
| (SEQ ID NO: 92) | VACVASGFTFRNFGMHWVRQAPGKGLEWVAFIWFDASN |
| | KGYGDSVKGRFTVSRDNSKNTLYLQMNGLRAEDTAVYY |
| | CAREKAVRGISRYNYYMDVWGKGTTVTVSSASTKGPSVF |
| | PLAPSSKSTSGGTAALGCLVKDYFPEPVTVSWNSGALTSG |
| | VHTFPAVLQSSGLYSLSSVVTVPSSSLGTQTYICNVNHKPS |
| | NTKVDKKAEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSN |
| | KALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSP |
| | GKGGGSIKQIEDKIEEILSKIYHIENEIARIKKLIGERGHERK |
| | CCVECPPCPEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSN |
| | KALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSP |
| | GK |
| GB4542 | METDTLLLWVLLLWVPGSTGQVQLQQPGAELVKPGASVK |
| (SEQ ID NO: 95) | MSCKASGYTFTSYNMHWVKQTPGRGLEWIGAIYPGNGDT |
| | SYNQKFKGKATLTADKSSSTAYMQLSSLTSEDSAVYYCA |
| | RSTYYGGDWYFNVWGAGTTVTVSAASTKGPSVFPLAPSS |
| | KSTSGGTAALGCLVKDYFPEPVTVSWNSGALTSGVHTFPA |
| | VLQSSGLYSLSSVVTVPSSSLGTQTYICNVNHKPSNTKVDK |
| | KAEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPI |
| | EKTISKAKGQPREPQVYTLPPSREEMTKNQVSLTCLVKGF |
| | YPSDIAVEWESNGQPENNYKTTPPVLDSDGSFFLYSKLTV |
| | DKSRWQQGNVFSCSVMHEALHNHYTQKSLSLSPGKGGGS |
| | IKQIEDKIEEILSKIYHIENEIARIKKLIGERGHERKCCVECPP |
| | CPEPKSCDKTHTCPPCPAPELLGGPSVFLFPPKPKDTLMISR |
| | TPEVTCVVVDVSHEDPEVKFNWYVDGVEVHNAKTKPREE |
| | QYNSTYRVVSVLTVLHQDWLNGKEYKCKVSNKALPAPIE |
| | KTISKAKGQPREPQVYTLPPSREEMTKNQVSLTCLVKGFY |
| | PSDIAVEWESNGQPENNYKTTPPVLDSDGSFFLYSKLTVD |
| | KSRWQQGNVFSCSVMHEALHNHYTQKSLSLSPGK |
| GB4542 Stable-GGGS | METDTLLLWVLLLWVPGSTGQVQLQQPGAELVKPGASVK |
| (SEQ ID NO: 96) | MSCKASGYTFTSYNMHWVKQTPGRGLEWIGAIYPGNGDT |
| | SYNQKFKGKATLTADKSSSTAYMQLSSLTSEDSAVYYCA |
| | RSTYYGGDWYFNVWGAGTTVTVSAASTKGPSVFPLAPSS |
| | KSTSGGTAALGCLVKDYFPEPVTVSWNSGALTSGVHTFPA |
| | VLQSSGLYSLSSVVTVPSSSLGTQTYICNVNHKPSNTKVDK |
| | KAEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPI |
| | EKTISKAKGQPREPQVYTLPPSREEMTKNQVSLTCLVKGF |
| | YPSDIAVEWESNGQPENNYKTTPPVLDSDGSFFLYSKLTV |
| | DKSRWQQGNVFSCSVMHEALHNHYTQKSLSLSPGKIKQIE |
| | DKIEEILSKIYHIENEIARIKKLIGERGHERKCCVECPPCPEP |
| | KSCDKTHTCPPCPAPELLGGPSVFLFPPKPKDTLMISRTPEV |
| | TCVVVDVSHEDPEVKFNWYVDGVEVHNAKTKPREEQYN |
| | STYRVVSVLTVLHQDWLNGKEYKCKVSNKALPAPIEKTIS |
| | KAKGQPREPQVYTLPPSREEMTKNQVSLTCLVKGFYPSDI |
| | AVEWESNGQPENNYKTTPPVLDSDGSFFLYSKLTVDKSR |
| | WQQGNVFSCSVMHEALHNHYTQKSLSLSPGK |
| GB4542 Stable-GH | METDTLLLWVLLLWVPGSTGQVQLQQPGAELVKPGASVK |
| (SEQ ID NO: 97) | MSCKASGYTFTSYNMHWVKQTPGRGLEWIGAIYPGNGDT |
| | SYNQKFKGKATLTADKSSSTAYMQLSSLTSEDSAVYYCA |
| | RSTYYGGDWYFNVWGAGTTVTVSAASTKGPSVFPLAPSS |
| | KSTSGGTAALGCLVKDYFPEPVTVSWNSGALTSGVHTFPA |
| | VLQSSGLYSLSSVVTVPSSSLGTQTYICNVNHKPSNTKVDK |
| | KAEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPI |
| | EKTISKAKGQPREPQVYTLPPSREEMTKNQVSLTCLVKGF |
| | YPSDIAVEWESNGQPENNYKTTPPVLDSDGSFFLYSKLTV |
| | DKSRWQQGNVFSCSVMHEALHNHYTQKSLSLSPGKGGGS |
| | IKQIEDKIEEILSKIYHIENEIARIKKLIGERERKCCVECPPCP |
| | EPKSCDKTHTCPPCPAPELLGGPSVFLFPPKPKDTLMISRTP |
| | EVTCVVVDVSHEDPEVKFNWYVDGVEVHNAKTKPREEQ |
| | YNSTYRVVSVLTVLHQDWLNGKEYKCKVSNKALPAPIEK |
| | TISKAKGQPREPQVYTLPPSREEMTKNQVSLTCLVKGFYPS |
| | DIAVEWESNGQPENNYKTTPPVLDSDGSFFLYSKLTVDKS |
| | RWQQGNVFSCSVMHEALHNHYTQKSLSLSPGK |
| GB4542 Stb-GGGSGH | METDTLLLWVLLLWVPGSTGQVQLQQPGAELVKPGASVK |
| (SEQ ID NO: 98) | MSCKASGYTFTSYNMHWVKQTPGRGLEWIGAIYPGNGDT |
| | SYNQKFKGKATLTADKSSSTAYMQLSSLTSEDSAVYYCA |
| | RSTYYGGDWYFNVWGAGTTVTVSAASTKGPSVFPLAPSS |
| | KSTSGGTAALGCLVKDYFPEPVTVSWNSGALTSGVHTFPA |
| | VLQSSGLYSLSSVVTVPSSSLGTQTYICNVNHKPSNTKVDK |
| | KAEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPI |
| | EKTISKAKGQPREPQVYTLPPSREEMTKNQVSLTCLVKGF |
| | YPSDIAVEWESNGQPENNYKTTPPVLDSDGSFFLYSKLTV |
| | DKSRWQQGNVFSCSVMHEALHNHYTQKSLSLSPGKIKQIE |
| | DKIEEILSKIYHIENEIARIKKLIGERERKCCVECPPCPEPKSC |
| | DKTHTCPPCPAPELLGGPSVFLFPPKPKDTLMISRTPEVTCV |
| | VVDVSHEDPEVKFNWYVDGVEVHNAKTKPREEQYNSTY |
| | RVVSVLTVLHQDWLNGKEYKCKVSNKALPAPIEKTISKAK |
| | GQPREPQVYTLPPSREEMTKNQVSLTCLVKGFYPSDIAVE |
| | WESNGQPENNYKTTPPVLDSDGSFFLYSKLTVDKSRWQQ |
| | GNVFSCSVMHEALHNHYTQKSLSLSPGK |
| GB10542 IL17 | METDTLLLWVLLLWVPGSTGEVQLVESGGGLVQPGGSLR |
| Stradobody | LSCAASGFTFSNYWMNWVRQAPGKGLEWVAAINQDGSE |
| (SEQ ID NO: 104) | KYYVGSVKGRFTISRDNAKNSLYLQMNSLRVEDTAVYYC |
| | VRDYYDILTDYYIHYWYFDLWGRGTLVTVSSASTKGPSVF |
| | PLAPSSKSTSGGTAALGCLVKDYFPEPVTVSWNSGALTSG |
| | VHTFPAVLQSSGLYSLSSVVTVPSSSLGTQTYICNVNHKPS |
| | NTKVDKRVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSN |
| | KALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSP |
| | GKGGGSIKQIEDKIEEILSKIYHIENEIARIKKLIGERGHERK |
| | CCVECPPCPEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSN |
| | KALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSP |
| | GK |
| GB11542 IL12/23 | METDTLLLWVLLLWVPGSTGEVQLVQSGAEVKKPGESLK |
| Stradobody | ISCKGSGYSFTTYWLGWVRQMPGKGLDWIGIMSPVDSDIR |
| (SEQ ID NO: 108) | YSPSFQGQVTMSVDKSITTAYLQWNSLKASDTAMYYCAR |
| | RRPGQGYFDFWGQGTLVTVSSSSTKGPSVFPLAPSSKSTSG |
| | GTAALGCLVKDYFPEPVTVSWNSGALTSGVHTFPAVLQSS |
| | GLYSLSSVVTVPSSSLGTQTYICNVNHKPSNTKVDKRVEP |
| | KSCDKTHTCPPCPAPELLGGPSVFLFPPKPKDTLMISRTPEV |
| | TCVVVDVSHEDPEVKFNWYVDGVEVHNAKTKPREEQYN |
| | STYRVVSVLTVLHQDWLNGKEYKCKVSNKALPAPIEKTIS |
| | KAKGQPREPQVYTLPPSREEMTKNQVSLTCLVKGFYPSDI |
| | AVEWESNGQPENNYKTTPPVLDSDGSFFLYSKLTVDKSR |
| | WQQGNVFSCSVMHEALHNHYTQKSLSLSPGKGGGSIKQIE |
| | DKIEEILSKIYHIENEIARIKKLIGERGHERKCCVECPPCPEP |
| | KSCDKTHTCPPCPAPELLGGPSVFLFPPKPKDTLMISRTPEV |
| | TCVVVDVSHEDPEVKFNWYVDGVEVHNAKTKPREEQYN |
| | STYRVVSVLTVLHQDWLNGKEYKCKVSNKALPAPIEKTIS |
| | KAKGQPREPQVYTLPPSREEMTKNQVSLTCLVKGFYPSDI |
| | AVEWESNGQPENNYKTTPPVLDSDGSFFLYSKLTVDKSR |
| | WQQGNVFSCSVMHEALHNHYTQKSLSLSPGK |
| GB4545 | METDTLLLWVLLLWVPGSTGQVQLQQPGAELVKPGASVK |
| (SEQ ID NO: 111) | MSCKASGYTFTSYNMHWVKQTPGRGLEWIGAIYPGNGDT |
| | SYNQKFKGKATLTADKSSSTAYMQLSSLTSEDSAVYYCA |
| | RSTYYGGDWYFNVWGAGTTVTVSAASTKGPSVFPLAPSS |
| | KSTSGGTAALGCLVKDYFPEPVTVSWNSGALTSGVHTFPA |
| | VLQSSGLYSLSSVVTVPSSSLGTQTYICNVNHKPSNTKVDK |
| | KAEPKSCDKTHTCPPCPAPELLGGPSVFLFPPKPKDTLMIS |
| | RTPEVTCVVVDVSHEDPEVKFNWYVDGVEVHNAKTKPRE |
| | EQYNSTYRVVSVLTVLHQDWLNGKEYKCKVSNKALPAPI |
| | EKTISKAKGQPREPQVYTLPPSREEMTKNQVSLTCLVKGF |
| | YPSDIAVEWESNGQPENNYKTTPPVLDSDGSFFLYSKLTV |
| | DKSRWQQGNVFSCSVMHEALHNHYTQKSLSLSPGKERKC |
| | CVECPPCP |
| GB9545 | METDTLLLWVLLLWVPGSTGQVKLLESGGGVVQPGGSLR |
| (SEQ ID NO: 112) | VACVASGFTFRNFGMHWVRQAPGKGLEWVAFIWFDASN |
| | KGYGDSVKGRFTVSRDNSKNTLYLQMNGLRAEDTAVYY |
| | CAREKAVRGISRYNYYMDVWGKGTTVTVSSASTKGPSVF |
| | PLAPSSKSTSGGTAALGCLVKDYFPEPVTVSWNSGALTSG |
| | VHTFPAVLQSSGLYSLSSVVTVPSSSLGTQTYICNVNHKPS |
| | NTKVDKKAEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSN |
| | KALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSP |
| | GKERKCCVECPPCP |
| GB10545 | METDTLLLWVLLLWVPGSTGEVQLVESGGGLVQPGGSLR |
| (SEQ ID NO: 113) | LSCAASGFTFSNYWMNWVRQAPGKGLEWVAAINQDGSE |
| | KYYVGSVKGRFTISRDNAKNSLYLQMNSLRVEDTAVYYC |
| | VRDYYDILTDYYIHYWYFDLWGRGTLVTVSSASTKGPSVF |
| | PLAPSSKSTSGGTAALGCLVKDYFPEPVTVSWNSGALTSG |
| | VHTFPAVLQSSGLYSLSSVVTVPSSSLGTQTYICNVNHKPS |
| | NTKVDKRVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSN |
| | KALPAPIEKTISKAKGQPREPQVYTLPPSREEMTKNQVSLT |
| | CLVKGFYPSDIAVEWESNGQPENNYKTTPPVLDSDGSFFL |
| | YSKLTVDKSRWQQGNVFSCSVMHEALHNHYTQKSLSLSP |
| | GKERKCCVECPPCP |
| GB11545 | METDTLLLWVLLLWVPGSTGEVQLVQSGAEVKKPGESLK |
| (SEQ ID NO: 114) | ISCKGSGYSFTTYWLGWVRQMPGKGLDWIGIMSPVDSDIR |
| | YSPSFQGQVTMSVDKSITTAYLQWNSLKASDTAMYYCAR |
| | RRPGQGYFDFWGQGTLVTVSSSSTKGPSVFPLAPSSKSTSG |
| | GTAALGCLVKDYFPEPVTVSWNSGALTSGVHTFPAVLQSS |
| | GLYSLSSVVTVPSSSLGTQTYICNVNHKPSNTKVDKRVEP |
| | KSCDKTHTCPPCPAPELLGGPSVFLFPPKPKDTLMISRTPEV |
| | TCVVVDVSHEDPEVKFNWYVDGVEVHNAKTKPREEQYN |
| | STYRVVSVLTVLHQDWLNGKEYKCKVSNKALPAPIEKTIS |
| | KAKGQPREPQVYTLPPSREEMTKNQVSLTCLVKGFYPSDI |
| | AVEWESNGQPENNYKTTPPVLDSDGSFFLYSKLTVDKSR |
| | WQQGNVFSCSVMHEALHNHYTQKSLSLSPGKERKCCVEC |
| | PPCP |

The skilled artisan will recognize that the specific stradobodies described above in Tables 1 and 2 are exemplary, and that stradobodies with various structures and combinations of stradomers and stradomer building blocks are useful in the compositions and methods of the present disclosure.

Antibodies comprise Fab domains from which a stradobody may be designed. Exemplary monoclonal antibodies from which a Fab domain for a stradobody may be designed include but are not limited to 3F8, 8H9, abagovomab, abciximab, adalimumab, adecatumumab, afelimomab, afutuzumab, alacizumab pegol, ALD518, alemtuzumab, altumomab pentetate, amatuximab, anatumomab mafenatox, anrukinzumab (IMA-638), apolizumab, arcitumomab, aselizumab, atinumab, atlizumab (tocilizumab), atorolimumab, bapineuzumab, basiliximab, bavituximab, bectumomab, belimumab, benralizumab, bertilimumab, besilesomab, bevacizumab, biciromab, bivatuzumab mertansine, blinatumomab, blosozumab, brentuximab vedotin, briakinumab, brodalumab, canakinumab, cantuzumab mertansine, cantuzumab ravtansine, capromab pendetide, carlumab, catumaxomab, CC49, cedelizumab, certolizumab pegol, cetuximab, Ch.14.18, citatuzumab bogatox, cixutumumab, clenoliximab, clivatuzumab tetraxetan, conatumumab, crenezumab, CR6261, dacetuzumab, daclizumab, dalotuzumab, daratumumab, denosumab, detumomab, dorlimomab aritox, drozitumab, ecromeximab, eculizumab, edobacomab, edrecolomab, efalizumab, efungumab, elotuzumab, elsilimomab, enavatuzumab, enlimomab pegol, enokizumab, ensituximab, epitumomab cituxetan, epratuzumab, erlizumab, ertumaxomab, etaracizumab, etrolizumab, exbivirumab, fanolesomab, faralimomab, farletuzumab, FBTA05, felvizumab, fezakinumab, ficlatuzumab, figitumumab, flanvotumab, fontolizumab, foralumab, foravirumab, fresolimumab, fulranumab, galiximab, ganitumab, gantenerumab, gavilimomab, gemtuzumab ozogamicin, gevokizumab, girentuximab, glembatumumab vedotin, golimumab, gomiliximab, GS6624, ibalizumab, ibritumomab tiuxetan, icrucumab, igovomab, imciromab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, lintuzumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-CD3, nacolomab tafenatox, namilumab, naptumomab estafenatox, narnatumab, natalizumab, nebacumab, necitumumab, nerelimomab, nimotuzumab, nofetumomab merpentan, obinutuzumab, ocrelizumab, odulimomab, ofatumumab, olaratumab, olokizumab, omalizumab, onartuzumab, oportuzumab monatox, oregovomab, otelixizumab, oxelumab, ozoralizumab, pagibaximab, palivizumab, panitumumab, panobacumab, pascolizumab, pateclizumab, pemtumomab, pertuzumab, pexelizumab, pintumomab, ponezumab, priliximab, pritumumab, PRO 140, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, siplizumab, sirukumab, solanezumab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, teneliximab, teplizumab, teprotumumab, TGN1412, ticilimumab (tremelimumab), tigatuzumab, TNX-650, tocilizumab (=atlizumab), toralizumab, tositumomab, tralokinumab, trastuzumab, TRBS07, tregalizumab, tremelimumab, tucotuzumab celmoleukin, tuvirumab, ublituximab, urelumab, urtoxazumab, ustekinumab, vapaliximab, vatelizumba, vedolizumab, veltuzumab, vepalimomab, vesencumab, visilizumab, volociximab, votumumab, zalutumumab, zanolimumab, ziralimumab, ZMapp anti-Ebola antibodies, zolimomab aritox, and monoclonal antibodies directed against Methicillin Resistant Staff Aureus, Vancomycin Resistant Enterococcus, Clostridium dificile, Mycobacterium tuberculosis, E coli 0157, and other infectious organisms and polyclonal antibodies directed against Rho(D).

The stradobody of the present invention may be specific for a cytokine. For example, the stradobody of the present invention may be specific for an Interferon (such as, for example, IFNγ, IFNα, or IFNβ), IL-1, IL-2, IL-4, IL-5, IL-6, IL-8, IL-10, IL-12, IL-13, IL-15, IL-17, or IL-23. In one embodiment, the stradobody of the current invention is specific for a cytokine, and is useful for treatment or prevention of one or more inflammatory diseases or autoimmune diseases. For example, in one embodiment, the stradobody is an anti-IL-2, anti-IL-8, or anti-IL-17 stradobody.

It is understood that the stradobodies disclosed herein can be derived from any of a variety of species. Indeed, Fc domains, or Fc partial domains, in any one biomimetic molecule of the present invention can be derived from immunoglobulin from more than one (e.g., from two, three, four, five, or more) species. However, they will more commonly be derived from a single species. In addition, it will be appreciated that any of the methods disclosed herein (e.g., methods of treatment) can be applied to any species. Generally, the components of a biomimetic applied to a species of interest will all be derived from that species. However, biomimetics in which all the components are of a different species or are from more than one species (including or not including the species to which the relevant method is applied) can also be used.

The specific CH1, CH2, CH3 and CH4 domains and hinge regions that comprise the Fc domains and Fc partial domains of the stradobodies of the present invention may be independently selected, both in terms of the immunoglobulin subclass, as well as in the organism, from which they are derived. Accordingly, the stradobodies disclosed herein may comprise Fc domains and partial Fc domains that independently come from various immunoglobulin types such as human IgG1, IgG2, IgG3, IgG4, IgA1, IgA2, IgD, IgE, and IgM, mouse IgG2a, or dog IgGa or IgGb. Preferably, for human therapeutics the Fc domains of the current invention are of the human IgG1 isotype. Similarly each Fc domain and partial Fc domain may be derived from various species, preferably a mammalian species, including non-human primates (e.g., monkeys, baboons, and chimpanzees), humans, murine, rattus, bovine, equine, feline, canine, porcine, rabbits, goats, deer, sheep, ferrets, gerbils, guinea pigs, hamsters, bats, birds (e.g., chickens, turkeys, and ducks), fish and reptiles to produce species-specific or chimeric stradobody molecules.

The Fab may be a chimeric structure comprised of human constant regions and non-human variable regions such as the variable region from a mouse, rat, rabbit, monkey, or goat antibody. One of ordinary skill in the art would be able to make a variety of Fab chimeric structures for incorporation into stradobodies using methodologies currently available and described in the scientific literature for such constructions. Individual Fab domains, Fc domains and partial Fc domains may also be humanized. Thus, “humanized” stradobodies may be designed analogous to “humanized” monoclonal antibodies.

Pharmaceutical Compositions

The route of administration of the stradobody compositions provided herein will vary, naturally, with the location and nature of the disease being treated, and may include, for example intradermal, transdermal, subdermal, parenteral, nasal, intravenous, intramuscular, subcutaneous, percutaneous, intratracheal, intraperitoneal, intratumoral, perfusion, lavage, direct injection, and oral administration. The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intratumoral, intraocular, nasal or intraarticular administration.

Such compositions would normally be administered as pharmaceutically acceptable compositions. The term “pharmaceutically acceptable composition” or “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. In a preferred embodiment the isolated stradobody is administered intravenously or subcutaneously.

Pharmaceutical stradobody compositions and methods for administering stradobody compositions, including sterile injectable compositions and compositions for other routes of administration, have been described in US 2014-0072582, incorporated herein by reference in its entirety for all purposes.

In addition, the stradobody of the current invention may optionally be administered before, during or after another pharmaceutical agent.

The stradobodies described herein may be administered at least once daily, weekly, biweekly or monthly or potentially less frequently. The stradobodies described herein may be administered at a dosing level designed to induce immune suppression as described herein. The stradobodies described herein may also be administered at two or more different dose levels depending on the intended effect of the stradobody. For example, in one embodiment, the stradobodies are administered at dose level intended to induce depletion of cells expressing the antigen to which the Fab region is directed. Because of the enhanced efficacy of the stradobodies of the current invention, in some embodiments the stradobodies may be administered at a lower dose intravenously compared with monoclonal antibodies specific for the same antigen. For example, the stradobodies may be administered at a first dose level that is lower than the optimal dose of a monoclonal antibody specific for the same antigen. In some embodiments, the first stradobody dose level is generally from about 1% to about 500% of the effective monoclonal antibody whose Fab is the same as the stradobody, more preferably, about 50% to about 100% of the effective monoclonal antibody dose. The effective monoclonal antibody dose in clinical cancer treatment varies. For the Her-2/neu monoclonal antibody, the dose is generally in the range of about 2 mg/Kg to about 4 mg/Kg administered every 7-21 days. For the EGFR monoclonal antibody the dose is generally in the range of about 250-400 mg/square meter which is about 5 mg/Kg-25 mg/Kg administered every 7-21 days. In another embodiment, the stradobodies are administered at a dose level intended to elicit an IVIG-like tolerogenic effect. In one embodiment, the stradobodies are administered at a first dose level followed by a second dose level. In some embodiments, the stradobodies are administered at a second dose level that is higher than the first dose level, wherein the second stradobody dose level induces an IVIG-like tolerogenic effect.

Therapeutic Applications of Stradobodies Having Bimodal Effects

The present inventors surprisingly found that at low doses, stradobodies function through their Fab domain, whereas at high doses they mimic the effector function of IVIG (or a stradomer), to mask the function of their Fab domain altogether. Without wishing to be bound by theory, the Fc function is so strong at high doses that the stradobody loses the antigen-specific binding activity of the Fab region. Thus, at low doses, stradobodies kill target cells or target infectious agents via the normal Fab domain-mediated killing mechanisms, whereas at high doses, the effector function of the Fc domain leads to tolerance. Thus, the stradobodies provided herein are useful for the treatment of inflammatory diseases, autoimmune diseases, cancers, or infectious diseases in which target cell killing or depletion followed by immune suppression to inhibit adverse inflammatory responses is desired.

The terms “treating” and “treatment” as used herein refer to administering to a subject a therapeutically effective amount of a stradobody of the present invention so that the subject has an improvement in a disease or condition, or a symptom of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. Specifically, improvements in subjects may include one or more of: decreased inflammation; decreased inflammatory laboratory markers such as C-reactive protein; decreased autoimmunity as evidenced by one or more of: improvements in autoimmune markers such as autoantibodies or in platelet count, white cell count, or red cell count, decreased rash or purpura, decrease in weakness, numbness, or tingling, increased glucose levels in patients with hyperglycemia, decreased joint pain, inflammation, swelling, or degradation, decrease in cramping and diarrhea frequency and volume, decreased angina, decreased tissue inflammation, or decrease in seizure frequency; decreases in cancer tumor burden, increased time to tumor progression, decreased cancer pain, increased survival or improvements in the quality of life; delay of progression or improvement of osteoporosis; or decreased symptoms of an infectious disease or decreased presence of an infectious agent (e.g., a decrease in viral load), and/or decrease in inflammation caused by immunopathogenicity triggered by an infectious agent (e.g., viral encephalitis, viral hemorrhagic fever, or sepsis).

In one embodiment, the present disclosure provides methods for reducing the incidence and/or severity of antibody mediated enhancement (AME). AME has been described in the art (Journal of Virology 77; 7539 (2003)) and develops when a subject develops antibodies against a virus during a virus infection. Virus-specific antibodies are then bound by C1q, enhancing internalization of the virus into cells, and thereby increasing viral load and worsening disease. In one embodiment, the stradobodies provided herein, which act as a complement sink, disrupt, prevent, or reduce AME by binding up C1q such that antibody-bound virus is not taken up by cells via C1q.

The term “therapeutically effective amount” or “effective amount” as used herein refers to an amount that results in an improvement or remediation of the symptoms of the disease or condition. In some embodiments, the therapeutically effective amount refers to different amounts, depending on the intended effect of the stradobody. For example, in one embodiment, a lower dose of stradobody is a therapeutically effective amount for depletion of target cells or killing of targeted infectious agents, and a higher dose of stradobody is a therapeutically effective amount for induction of immune suppression.

IVIG mediates immunosuppressive/tolerogenic activity. The precise mechanisms responsible for IVIG-like immunosuppression are not entirely understood, but are thought to include, without limitation, FcγR binding and blockage of antibody receptors on dendritic cells, monocytes, macrophages, B cells, and/or NK cells; enhanced complement-mediated removal of antibodies; and/or activation of regulatory T cells via T regulatory T cell epitopes present in the IVIG molecule. Through some or all of these mechanisms of action, IVIG reduces inflammation and/or induces immune tolerance. Thus, as used herein, the terms “IVIG-like effect” or “IVIG-like tolerance” and the like refer to anti-inflammatory, immunosuppressive, and tolerogenic effects similar to those mediated by IVIG. In some embodiments, a therapeutic dose of IVIG, or a therapeutically effective amount of IVIG, may be about 200 mg/kg, about 500 mg/kg, about 1 g/kg, about 2 g/kg, or more. In some embodiments, a therapeutic dose of IVIG is about 200 mg/kg to about 5 g/kg, or about 600 mg/kg to about 2 g/kg.

As used herein, “prophylaxis” can mean complete prevention of the symptoms of a disease, a delay in onset of the symptoms of a disease, or a lessening in the severity of subsequently developed disease symptoms.

The term “subject” is used interchangeably with the term “patient” herein, and is taken to mean any mammalian subject to which stradobodies of the present invention are administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish and reptiles.

In particular, the stradobodies of the present invention may be used to treat conditions including but not limited to congestive heart failure (CHF), vasculitis, rosacea, acne, eczema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; osteolytic bone cancers, lung cancer, kidney cancer and rectal cancer; bone metastasis, bone pain management, and humoral malignant hypercalcemia, ankylosing spondylitis and other spondyloarthropathies; transplantation rejection, viral infections, hematologic neoplasias and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histiocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, tumors of the vascular system (angiosarcoma and hemangiopericytoma)) or other cancer.

The stradobodies of the present invention may be used to treat antibody-mediated or non-antibody-mediated autoimmune diseases. The term “autoimmune disease” as used herein refers to a varied group of more than 80 diseases and conditions. In all of these diseases and conditions, the underlying problem is that the body's immune system attacks the body itself. Autoimmune diseases affect all major body systems including connective tissue, nerves, muscles, the endocrine system, skin, blood, and the respiratory and gastrointestinal systems. Autoimmune diseases include, for example, systemic lupus erythematosus, rheumatoid arthritis, multiple sclerosis, myasthenia gravis, and type 1 diabetes.

The disease or condition treatable using the compositions and methods of the present invention may be a hematoimmunological process, including but not limited to Idiopathic Thrombocytopenic Purpura, Pregnancy or delivery of an Rh-positive baby irrespective of the ABO groups of the mother and baby, Abortion/threatened abortion at any stage of gestation, Ectopic pregnancy, Antepartum fetal-maternal hemorrhage (suspected or proven) resulting from antepartum hemorrhage (e.g., placenta previa), amniocentesis, chorionic villus sampling, percutaneous umbilical blood sampling, other obstetrical manipulative procedure (e.g., version) or abdominal trauma Transfusion of Rh incompatible blood or blood products, alloimmune/autoimmune thrombocytopenia, Acquired immune thrombocytopenia, Autoimmune neutropenia, Autoimmune hemolytic anemia, Parvovirus B19-associated red cell aplasia, Acquired antifactor VIII autoimmunity, acquired von Willebrand disease, Multiple Myeloma and Monoclonal Gammopathy of Unknown Significance, Sepsis, Aplastic anemia, pure red cell aplasia, Diamond-Blackfan anemia, hemolytic disease of the newborn, Immune-mediated neutropenia, refractoriness to platelet transfusion, neonatal, post-transfusion purpura, hemolytic uremic syndrome, systemic Vasculitis, Thrombotic thrombocytopenic purpura, or Evan's syndrome.

The disease or condition may also be a neuroimmunological process, including but not limited to Guillain-Barre syndrome, Chronic Inflammatory Demyelinating Polyradiculoneuropathy, Paraproteinemic IgM demyelinating Polyneuropathy, Lambert-Eaton myasthenic syndrome, Myasthenia gravis, Multifocal Motor Neuropathy, Lower Motor Neuron Syndrome associated with anti-/GM1, Demyelination, Multiple Sclerosis and optic neuritis, Stiff Man Syndrome, Paraneoplastic cerebellar degeneration with anti-Yo antibodies, paraneoplastic encephalomyelitis, sensory neuropathy with anti-Hu antibodies, epilepsy, Encephalitis, Myelitis, Myelopathy especially associated with Human T-cell lymphotropic virus-1, Autoimmune Diabetic Neuropathy, Alzheimer's disease, Parkinson's disease, Huntingdon's disease, or Acute Idiopathic Dysautonomic Neuropathy.

The disease or condition may also be a Rheumatic disease process, including but not limited to Kawasaki's disease, Rheumatoid arthritis, Felty's syndrome, ANCA-positive Vasculitis, Spontaneous Polymyositis, Dermatomyositis, Antiphospholipid syndromes, Recurrent spontaneous abortions, Systemic Lupus Erythematosus, Juvenile idiopathic arthritis, Raynaud's, CREST syndrome, or Uveitis.

The disease or condition may also be a dermatoimmunological disease process, including but not limited to Toxic Epidermal Necrolysis, Gangrene, Granuloma, Autoimmune skin blistering diseases including Pemphigus vulgaris, Bullous Pemphigoid, Pemphigus foliaceus, Vitiligo, Streptococcal toxic shock syndrome, Scleroderma, systemic sclerosis including diffuse and limited cutaneous systemic sclerosis, or Atopic dermatitis (especially steroid dependent).

The disease or condition may also be a musculoskeletal immunological disease process, including but not limited to Inclusion Body Myositis, Necrotizing fasciitis, Inflammatory Myopathies, Myositis, Anti-Decorin (BJ antigen) Myopathy, Paraneoplastic Necrotic Myopathy, X-linked Vacuolated Myopathy, Penacillamine-induced Polymyositis, Atherosclerosis, Coronary Artery Disease, or Cardiomyopathy.

The disease or condition may also be a gastrointestinal immunological disease process, including but not limited to pernicious anemia, autoimmune chronic active hepatitis, primary biliary cirrhosis, Celiac disease, dermatitis herpetiformis, cryptogenic cirrhosis, Reactive arthritis, Crohn's disease, Whipple's disease, ulcerative colitis, or sclerosing cholangitis.

The disease or condition may also be Graft Versus Host Disease, Antibody-mediated rejection of the graft, Post-bone marrow transplant rejection, Post-infectious disease inflammation, Lymphoma, Leukemia, Neoplasia, Asthma, Type 1 Diabetes mellitus with anti-beta cell antibodies, Sjogren's syndrome, Mixed Connective Tissue Disease, Addison's disease, Vogt-Koyanagi-Harada Syndrome, Membranoproliferative glomerulonephritis, Goodpasture's syndrome, Graves' disease, Hashimoto's thyroiditis, Wegener's granulomatosis, micropolyarterits, Churg-Strauss syndrome, Polyarteritis nodosa or Multisystem organ failure.

In addition to having clinical utility for treating immunological disorders, stradobodies have therapeutic use in infectious disease, cancer, and inflammatory disease treatment. The stradobodies may be used essentially following known protocols for any corresponding therapeutic antibody, and have the advantage not only of enhanced potency relative to the corresponding therapeutic antibody, but also the added advantage of IVIG-like immune suppression.

Infectious diseases, include, but are not limited to, those caused by bacterial, mycological, parasitic, and viral agents. Examples of such infectious agents include the following: staphylococcus, streptococcaceae, neisseriaaceae, cocci, enterobacteriaceae, pseudomonadaceae, vibrionaceae, campylobacter, pasteurellaceae, bordetella, francisella, brucella, legionellaceae, bacteroidaceae, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, other gram-positive bacilli, other gram-negative bacilli, systemic mycoses, other opportunistic mycoses, protozoa, nematodes, trematodes, cestodes, adenoviruses, herpesviruses (including, for example, herpes simplex virus and Epstein Barr virus, and herpes zoster virus), poxviruses, papovaviruses, hepatitis viruses, papilloma viruses, orthomyxoviruses (including, for example, influenza A, influenza B, and influenza C), paramyxoviruses, coronaviruses, picornaviruses, reoviruses, togaviruses (e.g., alpha viruses such as Chikungunya virus), filoviruses (e.g., Ebolavirus, Margurgvirus, and Cuevavirus), flaviviruses (e.g., West Nile virus, Dengue virus, Yellow Fever virus, and Japanese Encephalitis virus), bunyaviridae, rhabdoviruses, respiratory syncitial virus, human immunodeficiency virus and retroviruses. Exemplary infectious diseases include but are not limited to candidiasis, candidemia, aspergillosis, streptococcal pneumonia, streptococcal skin and oropharyngeal conditions, gram negative sepsis, tuberculosis, mononucleosis, influenza, respiratory illness caused by Respiratory Syncytial Virus, malaria, Ebola virus disease (also known as Ebola hemorrhagic fever), encephalitis, schistosomiasis, and trypanosomiasis.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, lymphangiosarcoma, lymphangioendotheliosarcoma, leiomyosarcoma, rhabdomyosarcoma, fibrosarcoma, myxosarcoma, chondrosarcoma), osteoclastoma, neuroendocrine tumors, mesothelioma, chordoma, synovioma, schwanoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwanoma, and other carcinomas, head and neck cancer, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, tumors of the vascular system (angiosarcoma and hemangiopericytoma), hematologic neoplasias and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, osteolytic bone cancers, and bone metastasis.

The term “autoimmune disease” as used herein refers to a varied group of more than 80 chronic illnesses. In all of these diseases, the underlying problem is that the body's immune system attacks the body itself. Autoimmune diseases affect all major body systems including connective tissue, nerves, muscles, the endocrine system, skin, blood, and the respiratory and gastrointestinal systems.

The autoimmune disease or condition may be a hematoimmunological process, including but not limited to Idiopathic Thrombocytopenic Purpura, alloimmune/autoimmune thrombocytopenia, Acquired immune thrombocytopenia, Autoimmune neutropenia, Autoimmune hemolytic anemia, Parvovirus B19-associated red cell aplasia, Acquired antifactor VIII autoimmunity, acquired von Willebrand disease, Multiple Myeloma and Monoclonal Gammopathy of Unknown Significance, Sepsis, Aplastic anemia, pure red cell aplasia, Diamond-Blackfan anemia, hemolytic disease of the newborn, Immune-mediated neutropenia, refractoriness to platelet transfusion, neonatal, post-transfusion purpura, hemolytic uremic syndrome, systemic Vasculitis, Thrombotic thrombocytopenic purpura, or Evan's syndrome.

The autoimmune disease or condition may be a neuroimmunological process, including but not limited to Guillain-Barre syndrome, Chronic Inflammatory Demyelinating Polyradiculoneuropathy, Paraproteinemic IgM demyelinating Polyneuropathy, Lambert-Eaton myasthenic syndrome, Myasthenia gravis, Multifocal Motor Neuropathy, Lower Motor Neuron Syndrome associated with anti-/GM1, Demyelination, Multiple Sclerosis and optic neuritis, Stiff Man Syndrome, Paraneoplastic cerebellar degeneration with anti-Yo antibodies, paraneoplastic encephalomyelitis, sensory neuropathy with anti-Hu antibodies, epilepsy, Encephalitis, Myelitis, Myelopathy especially associated with Human T-cell lymphotropic virus-1, Autoimmune Diabetic Neuropathy, or Acute Idiopathic Dysautonomic Neuropathy.

The autoimmune disease or condition may be a Rheumatic disease process, including but not limited to Kawasaki's disease, Rheumatoid arthritis, Felty's syndrome, ANCA-positive Vasculitis, Spontaneous Polymyositis, Dermatomyositis, Antiphospholipid syndromes, Recurrent spontaneous abortions, Systemic Lupus Erythematosus, Juvenile idiopathic arthritis, Raynaud's, CREST syndrome, or Uveitis.

The autoimmune disease or condition may be a dermatoimmunological disease process, including but not limited to Toxic Epidermal Necrolysis, Gangrene, Granuloma, Autoimmune skin blistering diseases including Pemphigus vulgaris, Bullous Pemphigoid, and Pemphigus foliaceus, Vitiligo, Streptococcal toxic shock syndrome, Scleroderma, systemic sclerosis including diffuse and limited cutaneous systemic sclerosis, or Atopic dermatitis (especially steroid dependent).

The autoimmune disease or condition may be a gastrointestinal immunological disease process, including but not limited to pernicious anemia, autoimmune chronic active hepatitis, primary biliary cirrhosis, Celiac disease, dermatitis herpetiformis, cryptogenic cirrhosis, Reactive arthritis, Crohn's disease, Whipple's disease, ulcerative colitis, or sclerosing cholangitis.

The autoimmune disease or condition may be Graft Versus Host Disease, Antibody-mediated rejection of the graft, Post-bone marrow transplant rejection, Post-infectious disease inflammation, Lymphoma, Leukemia, Neoplasia, Asthma, Type 1 Diabetes mellitus with anti-beta cell antibodies, Sjogren's syndrome, Mixed Connective Tissue Disease, Addison's disease, Vogt-Koyanagi-Harada Syndrome, Membranoproliferative glomerulonephritis, Goodpasture's syndrome, Graves' disease, Hashimoto's thyroiditis, Wegener's granulomatosis, micropolyarterits, Churg-Strauss syndrome, Polyarteritis nodosa or Multisystem organ failure.

The stradobodies disclosed herein have a number of further applications and uses.

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the terms “biomimetic”, “biomimetic molecule”, “biomimetic compound”, and related terms, may refer to a human made compound that imitates the function of another compound, such as pooled human Intravenous Immunoglobulin (“IVIG”), a monoclonal antibody or the Fc or Fab fragment of an antibody. “Biologically active” biomimetics are compounds which possess biological activities that are the same as or similar to their naturally occurring counterparts. By “naturally occurring” is meant a molecule or portion thereof that is normally found in an organism. By naturally occurring is also meant substantially naturally occurring. “Immunologically active” biomimetics are biomimetics which exhibit immunological activity the same as or similar to naturally occurring immunologically active molecules, such as antibodies, cytokines, interleukins and other immunological molecules known in the art. In preferred embodiments, the biomimetics of the present invention are stradobodies, as defined herein. A “bimodal” stradobody, as used herein, refers to a stradobody having two different modes of action. Specifically, in some embodiments, the bimodal stradobodies provided herein exhibit both antibody-like Fc functions and IVIG-like immune suppression.

By “homologous” is meant identity over the entire sequence of a given nucleic acid or amino acid sequence. For example, by “80% homologous” is meant that a given sequence shares about 80% identity with the claimed sequence and can include insertions, deletions, substitutions, and frame shifts. One of ordinary skill in the art will understand that sequence alignments can be done to take into account insertions and deletions to determine identity over the entire length of a sequence.

All references, patents, and patent applications cited herein are incorporated in their entirety for all purposes.

The following examples are provided by way of illustration only and not by way of limitation. Those of skill in the art will readily recognize a variety of non-critical parameters that could be changed or modified to yield essentially similar results.

### Examples

### Example 1. Bimodal Effector Function of Stradobodies In Vitro

To determine if an anti-CD20 stradobody could engage with FcγRs and complement, independent of Fab-antigen interactions, we tested the binding and activity of the stradobody to different cell types and at different dose levels.

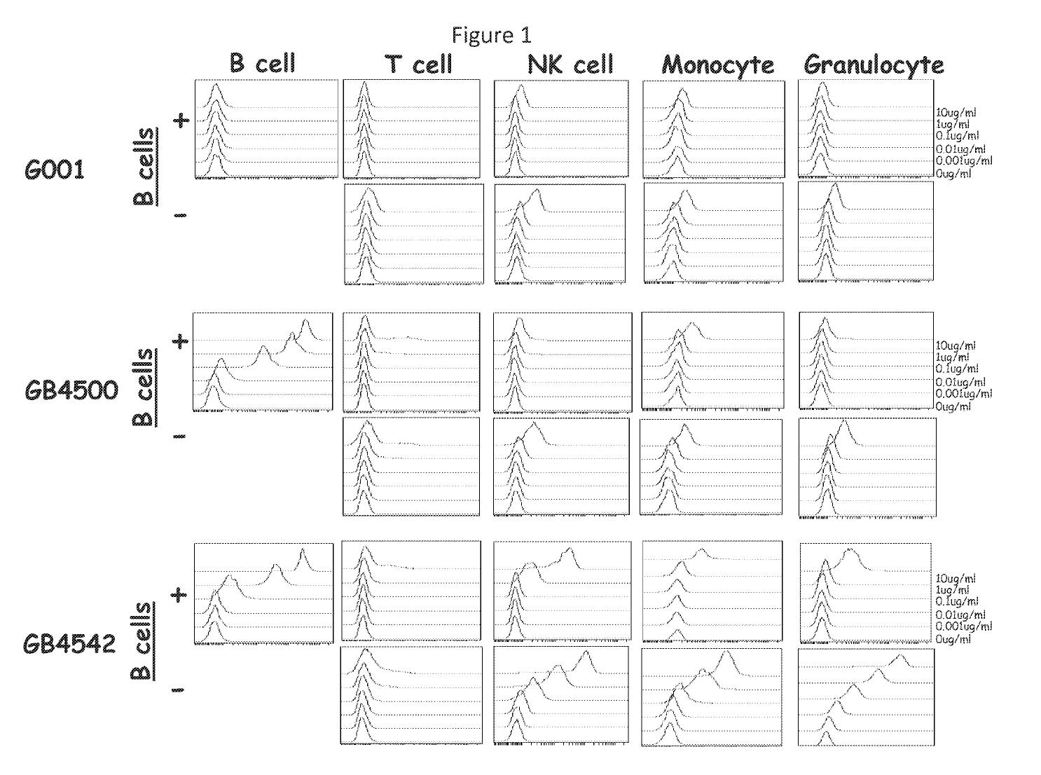

First, binding of G001 (negative control), GB4500 (parent CD20 antibody comprising the rituximab Fab), or GB4542 (comprising the identical rituximab Fab) to human B cells, or to human T cells, NK cells, monocytes, or granulocytes in the presence or absence of human B cells, was tested at 0, 0.001, 0.01, 0.1, 1, or 10 μg/ml. The results of the study are shown in FIG. 1. GB4500 and GB4542 each exhibited dose-dependent binding of B cells, and importantly a preferential binding to B cells over other cell types. GB4542, but not GB4500, exhibited some binding to NK cells, monocytes, and granulocytes even in the presence of B cells, at doses higher than 1 μg/ml. When B cells were not present, GB4542, but not GB4500, exhibited binding to NK cells, monocytes, and granulocytes at moderate to high doses (e.g., doses of 0.1, 1, and 10 μg/ml). The results of this study showed that GB4542 preferentially binds B cells, but is also capable of binding FcγR expressing cells to a much higher degree relative to GB4500.

Next, the effector functions of CD20-specific stradobody GB4542 over a range of doses and relative to the parent CD20 antibody, GB4500 (rituximab) were tested. Serial dilutions of GB4542, GB4500, or IVIG were incubated with donor human cells to determine ADCC, ADCP, and CDC activity.

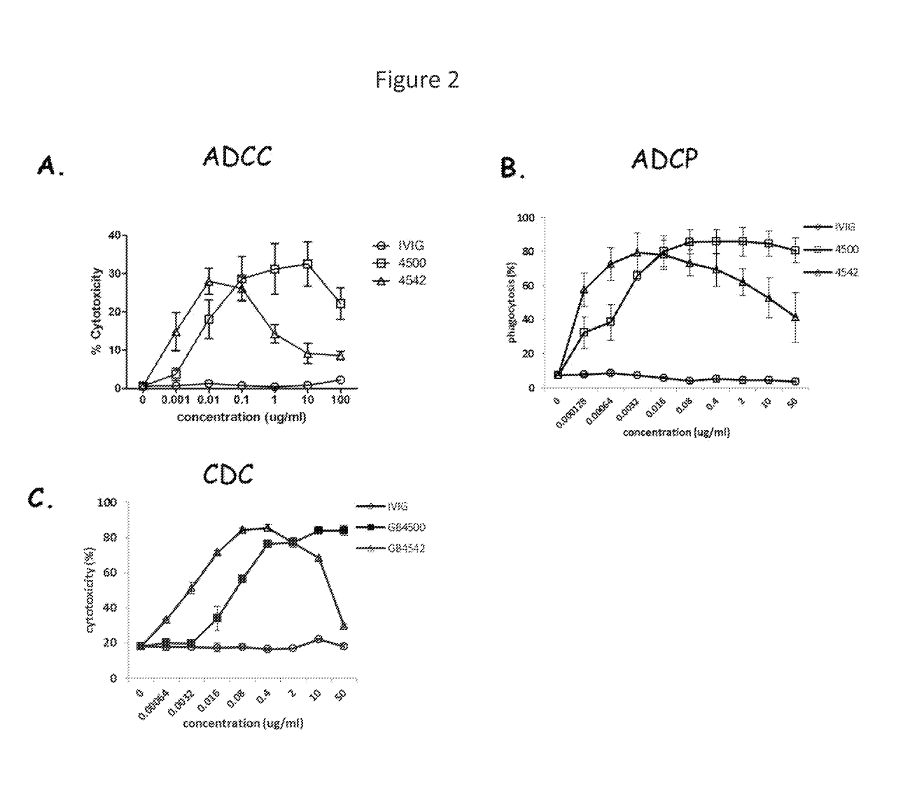

FIG. 2 provides the results of the ADCC, ADCP, and CDC studies. FIG. 2A shows that at low doses, GB4542 exhibited a dose-response ADCC profile, but that at higher concentrations, GB4542 exhibited suppressive and/or ineffective B cell killing. FIGS. 2B and 2C, respectively, show similar bimodal activity of GB4542 with respect to ADCP (expressed as percent phagocytosis) and CDC activity. In contrast, parent antibody GB4500 exhibited a typical dose dependent fashion at lower concentrations with a subsequent plateau, for each assay. IVIG did not exhibit ADCC, ADCP, or CDC. In addition, the optimal concentration of GB4542 required for ADCC, ADCP and CDC was about one log order lower than GB4500, indicating that the stradobody exhibits more potent FcγR cross-linking on effector immune cells and complement activation relative to the parent monoclonal antibody.

Thus, the results of the study unexpectedly showed, while low doses of the stradobody mediated potent B cell killing in vitro, resulting from enhanced ADCC, ADCP, and CDC, higher doses protected B cells from depletion and inhibited phagocytosis.

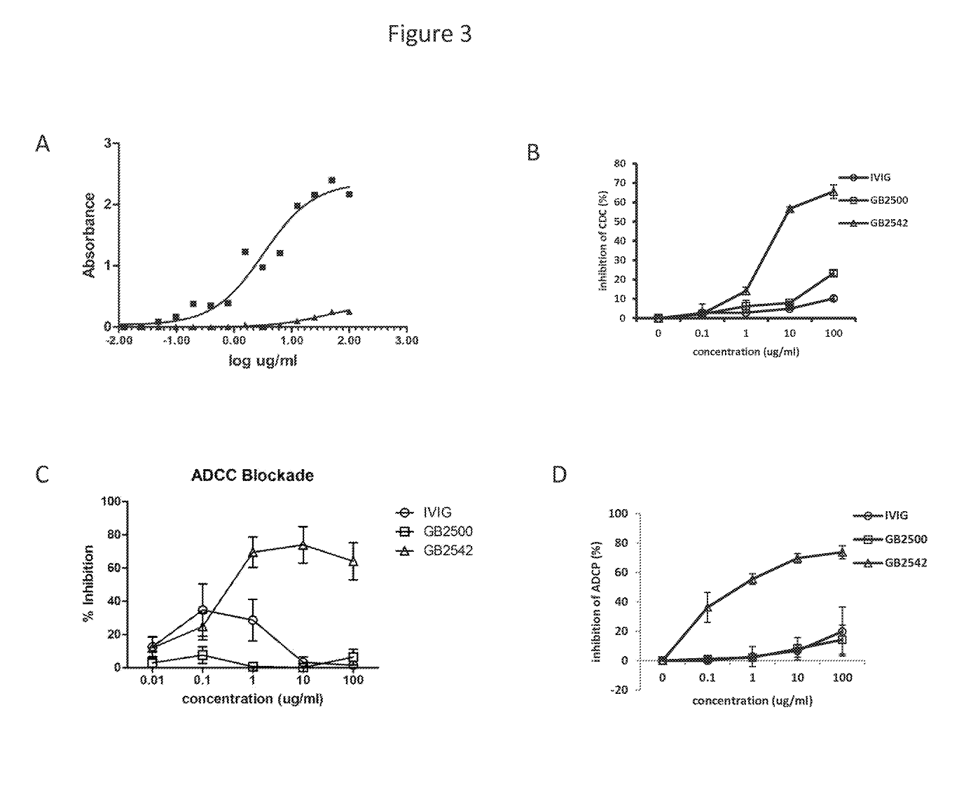

In order to understand how GB4542 mediates complement dependent B cell killing at “low” doses yet protects B cells from CDC at “higher” doses, we first analyzed the ability of GB4542 and GB4500 to bind C1q. Consistent with the documented ability of Ab opsonized cells to engage C1q, the first step in classical complement activation, GB4542 bound C1q in a dose dependent fashion (FIG. 3A). In contrast, GB4500 evidenced negligible interactions with C1q, even at the highest doses tested. Next, to determine if the multimerized Fc fragments in GB4542 could protect B cells from CDC, we tested the ability of GB2542 (a stradobody having an Fab specific for Her2/neu antigen) to block GB4500-mediated CDC of B cells (FIG. 3B). Interestingly, GB2542 protected B cells from GB4500 mediated CDC. Collectively, these data indicated that GB4542, at higher doses, can bind C1q and inhibit mAb mediated CDC, likely, but not definitively, by engaging complement away from the surface of CD20⁺ cells. Next, we employed an analogous strategy to determine if addition of GB2542 could interfere with Fc:FcγR mediated ADCC and ADCP. (FIG. 3C, D) Consistent with our complement data, we observed that GB2542 protected B cells from both ADCC and ADCP at higher doses. Importantly, the doses at which these protective effects in CDC, ADCC and ADCP observed were consistent with the inflection point at which the ability of GB4542 to mediate these effector functions began to decline.

These data demonstrated that at higher doses, the multimerized Fc “tails” of stradobodies inhibit complement mediated lysis, block NK cell ADCC and dampen macrophage phagocytosis of human B cells, independent of Fab specificity.

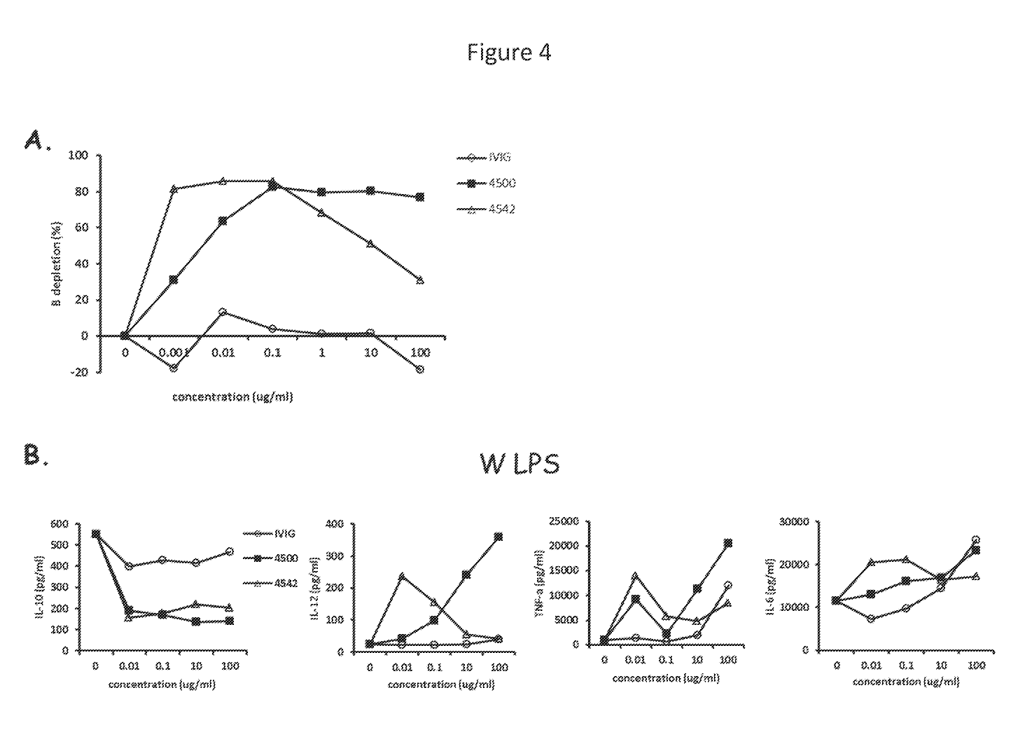

Next, we studied the ability of GB4542 to mediate B cell depletion in PBMC. At concentrations ≤0.1 μg/ml, GB4542 mediated a dose dependent B cell depletion, which was approximately 1-log order more potent than GB4500 at all doses tested. (FIG. 4A) In contrast, at concentrations above 0.1 μg/ml, while GB4500 maintained its capacity for B cell depletion, increasing doses of GB4542 were inversely correlated with B cell loss. Importantly, IVIG, used as a control for homodimeric and aggregated Fc domains, did not mediate appreciable B cell reduction.

In addition, we tested whether GB4500 and/or GB4542 induced a pro-inflammatory cytokine response in PBMC. In the absence of LPS, neither drug mediated appreciable cytokine release. However, in the presence of LPS, used as a surrogate for systemic inflammation, both GB4500 and GB4542 stimulated both IL-12 and TNF release (FIG. 4B). The presence of these cytokines was directly correlated with the concentrations of GB4500 and GB4542 required to mediate optimal B cell depletion, with GB4542 stimulating cytokine production at lower concentrations and limited induction of cytokine release at higher doses. In contrast, GB4500 continued to stimulate increasing levels of pro-inflammatory cytokine release at concentrations higher than those required for optimal B cell depletion, indicating that the stradobody exhibited bimodal activity with respect to the induction of inflammatory cytokine release.

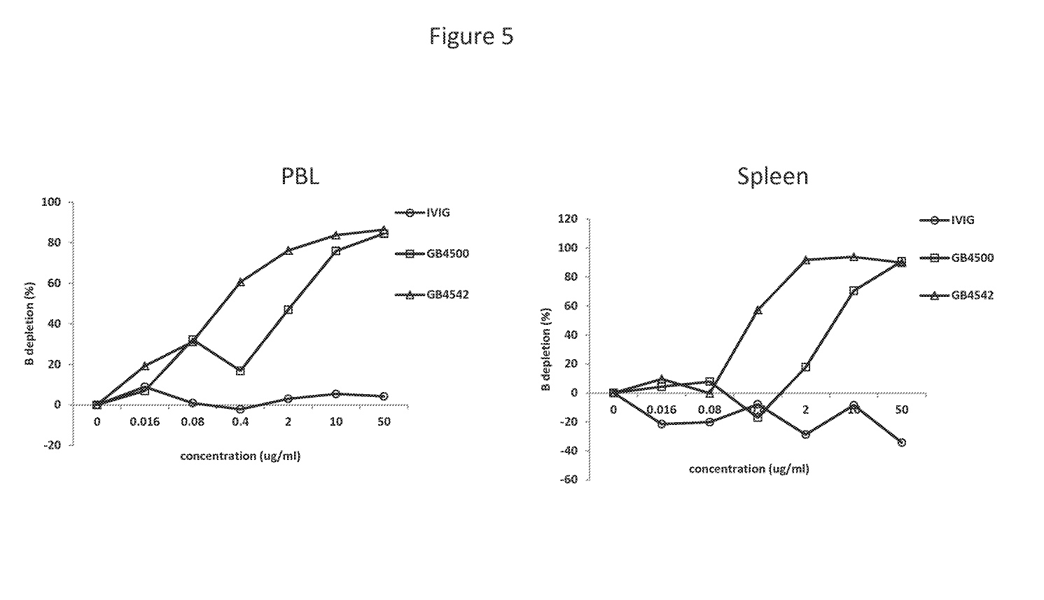

Next we tested whether similar effects were seen in lower order species. Peripheral blood and spleen were obtained from cynomolgus monkeys, assessed for CDC activity induced by a range of concentrations of GB4500, GB4542, and IVIG. PBMC and spleen cells were cultured with serial dilutions of anti-human CD20 monoclonal antibody or stradobody in the presence of 10% cynomolgus serum (as a source of complement) for 1 hour. B cells were gated as CD3-DR+ lymphocytes. Cell apoptosis/death was determined by Annexin V/7-AAD staining. The data are presented in FIG. 5, and are shown as percent of B cell depletion relative to no treatment control. As demonstrated in FIG. 5, GB4542 induced an enhanced CDC activity compared with GB4500 within the dose ranges: 0.4-10 μg/ml but a higher concentration of drug is required to generate Complement Dependent Cytotoxicity relative to human blood, suggesting that GB4542 may be even more potent in human than in monkey. The tolerogenic effect has not been demonstrated at concentrations up to 50 μg/ml but may exist at higher concentrations. Similarly, a murine macrophage phagocytosis assay demonstrated significantly less potency of GB4542 relative to a human macrophage phagocytosis assay.

### Example 2. In Vivo Stradobody-Mediated B Cell Depletion

In vivo studies in mice with high doses, such as 20-40 mg/Kg, of anti-CD20 stradobody GB4542 in xenotransplant studies failed to mediate depletion of B cells. A study was conducted to determine if low doses of GB4542 mediate B cell depletion in the peripheral blood of monkeys. Cynomolgus monkeys (having an approximate circulating blood volume of 65 mL/kg) were administered 0.1 mg/kg (100 μg/kg) or 1.0 mg/kg (1000 μg/kg) GB4542 as shown below in Table 3.

TABLE 3
GB4542 dosing strategy for monkey experiments

| | Drug/mL based on | |
|---|---|---|
| Dose | 65 mL/kg blood volume | Dosing regimen |
| 0.1 mg/kg | 1.54 μg/mL | 0.1 mg/kg over 1 hour |
| 1.0 mg/kg | 15.4 μg/mL | 0.1 mg/kg in the first |
| | | 10 minutes, 10 minute break, |
| | | remaining dose over the |
| | | following 40 minutes |

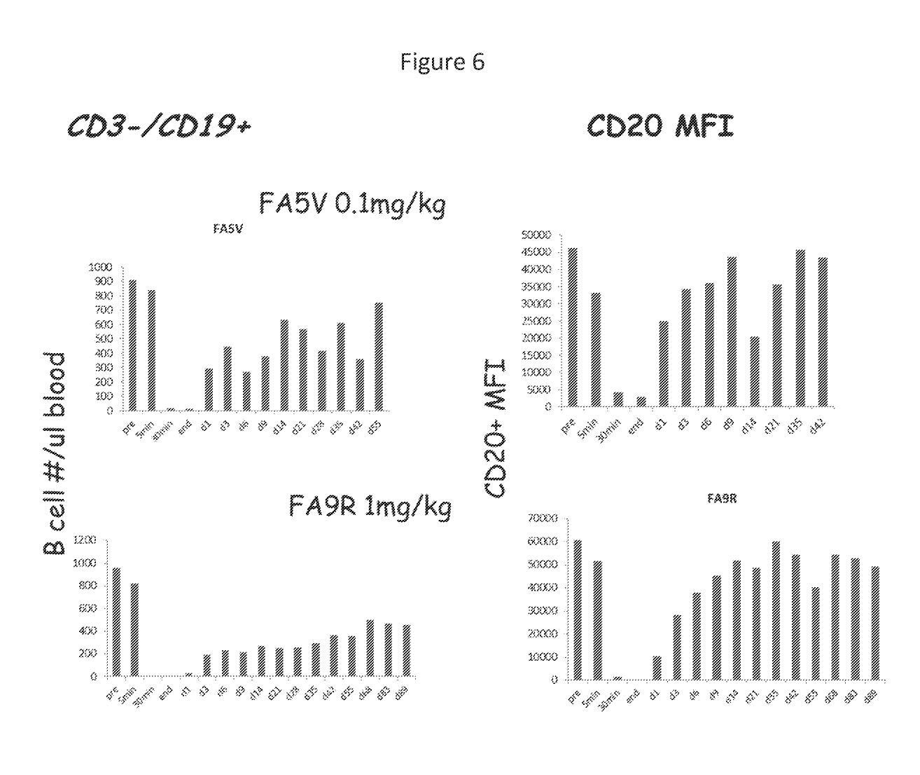

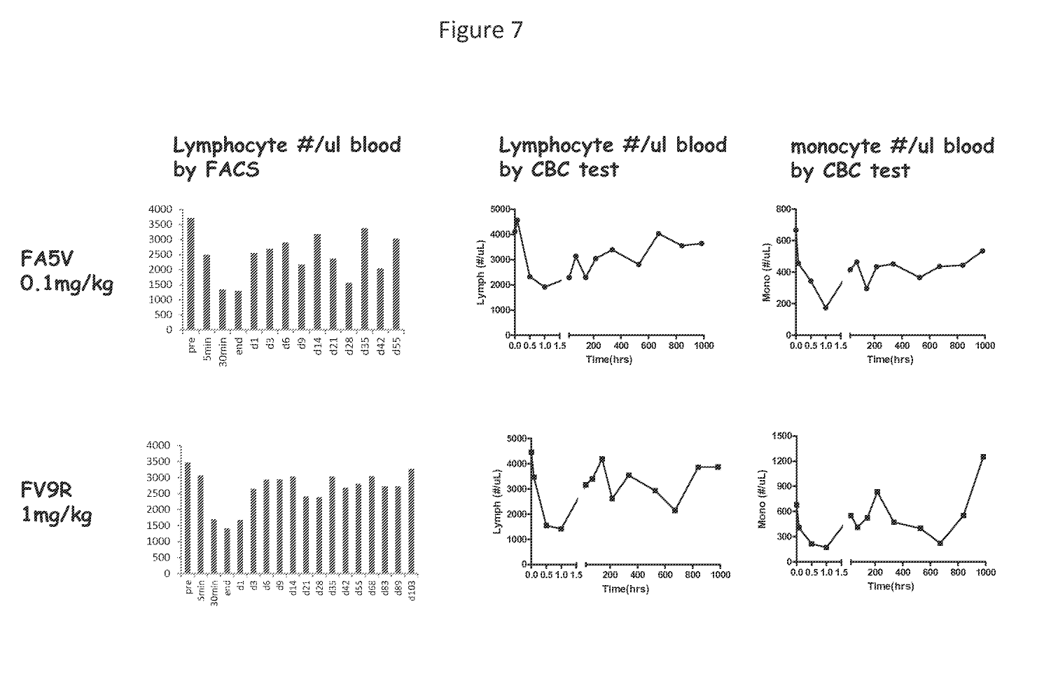

The results of the study are shown in FIGS. 6 and 7. Both 0.1 mg/kg and 1.0 mg/kg doses resulted in depletion of B cells in the blood as measured by CD3-CD19+ B cell number per μL blood (FIG. 6, left panels) as well as by mean fluorescence intensity (MFI) of CD20 (FIG. 6, right panels). FIG. 7 provides the number of lymphocytes and monocytes per μL blood for the 0.1 mg/kg dose (top three panels) and 1.0 mg/kg dose (bottom three panels). Both dosing levels mediated complete B cell depletion and/or sequestration and decreased and/or blocked CD20 levels during the infusion. After infusion, the total number of lymphocytes and monocytes in the peripheral blood was decreased transiently, and B cell depletion was sustained in the periphery at the 1.0 mg/kg dose. Given the lack of activity of GB4542 in vivo in mice, the activity of GB4542 in vivo in monkeys was particularly surprising.

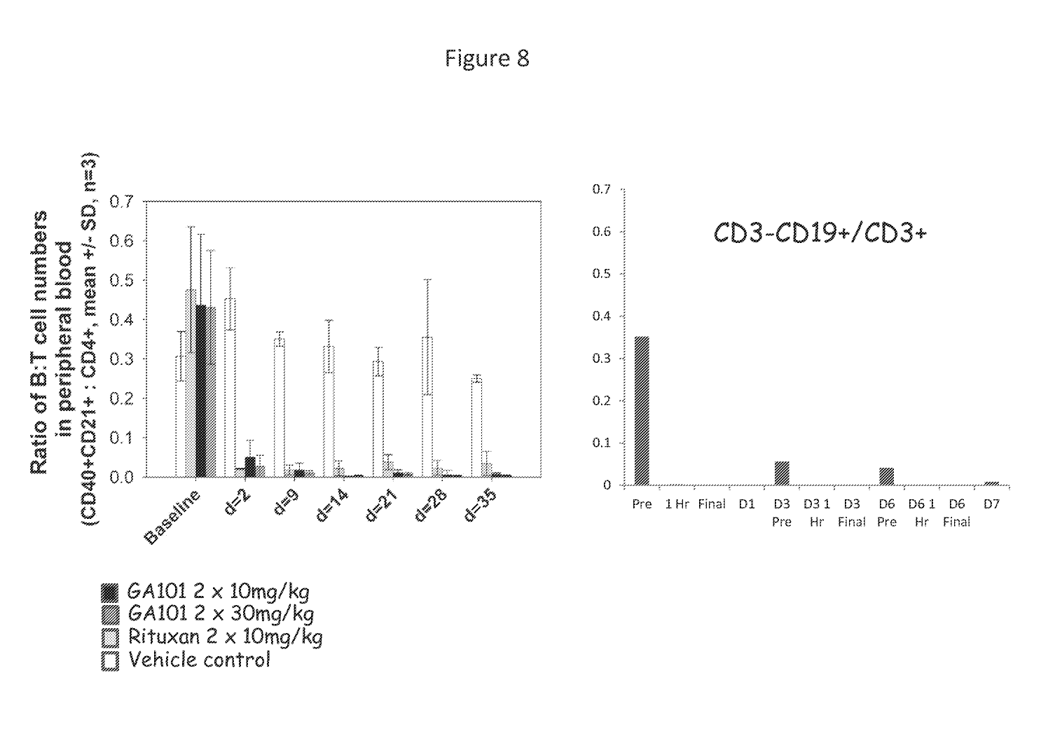

To test the effect of repetitive low doses of GB4542 in monkeys, a dose of 1 mg/kg GB4542 was administered every three days for three total doses. Specifically, 0.1 mg/kg was administered over 1 hour, and then the remaining 0.9 mg/kg was administered over the next hour at each dosing time point. Monkeys remained asymptomatic. The results of the study are provided in FIG. 8, right panel. Depletion of CD3-CD19+ B cells in the blood was achieved and maintained for at least 7 days. The left panel of FIG. 8 shows that rituximab (Rituxan; 2 doses of 10 mg/kg) or obinutuzumab (a humanized anti-CD20 antibody; GA101; 2 doses of 10 mg/kg or 30 mg/kg) both deplete B cells in cynomolgus monkeys.

The study indicated that GB4542 depletes B cells in vivo at least as well as rituximab or s obinutuzumab, and may deplete B cells at much lower doses than rituximab or obinutuzumab, demonstrating that low doses of GB4542 may be more potent than anti-CD20 monoclonal antibodies including rituximab, which comprises the identical anti-CD20 Fab.

### Example 3. In Vivo Dose Response of GB4542: Effect on B Cell Depletion

GB4542 is administered to animals (e.g., cynomolgus monkeys or mice) by subcutaneous administration at doses of, for example, 0.1, 0.5, 1, or 10 mg/kg at day 0. Blood is drawn, for example at days 1, 4, 7, and 14 after treatment and analyzed by flow cytometry for B cell depletion. The study will show that animals that receive lower doses of GB4542 exhibit more B cell depletion than animals that receive higher doses of GB4542. For example, animals that receive the highest dose of 10 mg/kg exhibit less B cell depletion relative to animals that receive the lowest dose of 0.1 mg/kg.

### Example 4: In Vivo Dose Response of GB4542: Effect on Tolerance

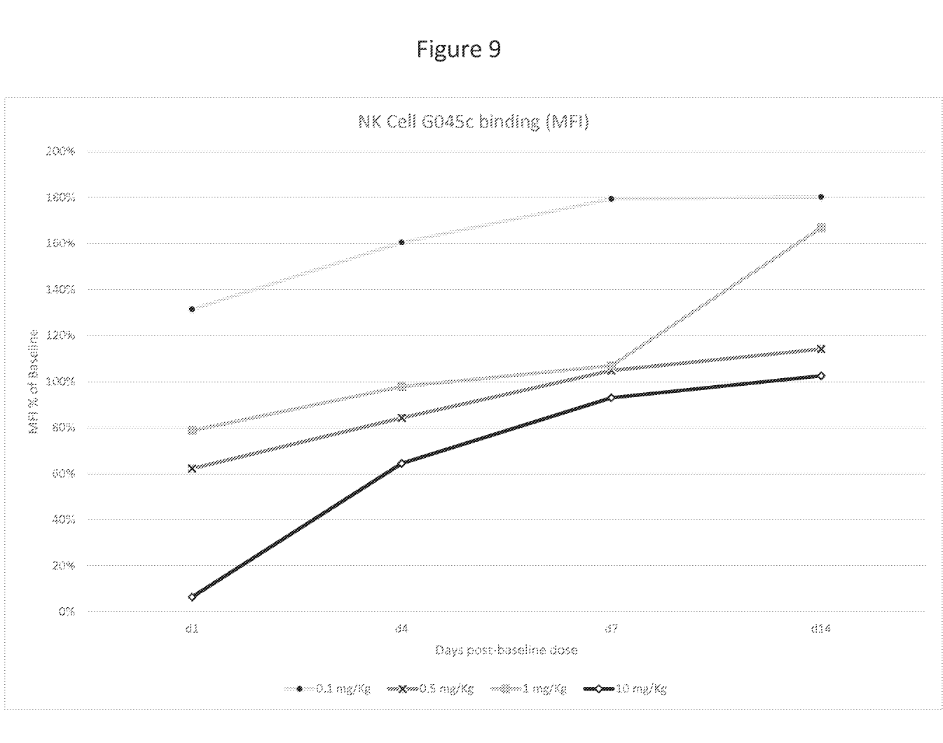

GB4542 was administered to cynomolgus monkeys by subcutaneous administration at a dose of 0.1, 0.5, 1, or 10 mg/kg at day 0. Blood was drawn at days 1, 4, 7, and 14 after treatment and analyzed by flow cytometry for FcγR blocking/downregulation shown by the level of G045c binding with FcγR expressing cells (NK cells). In animals that received the highest dose of 10 mg/kg, NK cells exhibited less G045c binding compared to G045c binding in animals that received the lowest dose of 0.1 mg/kg (FIG. 9). Without wishing to be bound by theory, FcγRIII on NK cells may be down-modulated or blocked following administration of a high dose of GB4542, resulting in decreased availability of FcγRIII for GO45c binding

### Example 5. Higher Molecular Weight Fraction of GB4542 Mediates Reduced B Cell Depletion In Vivo in Non-Human Primates

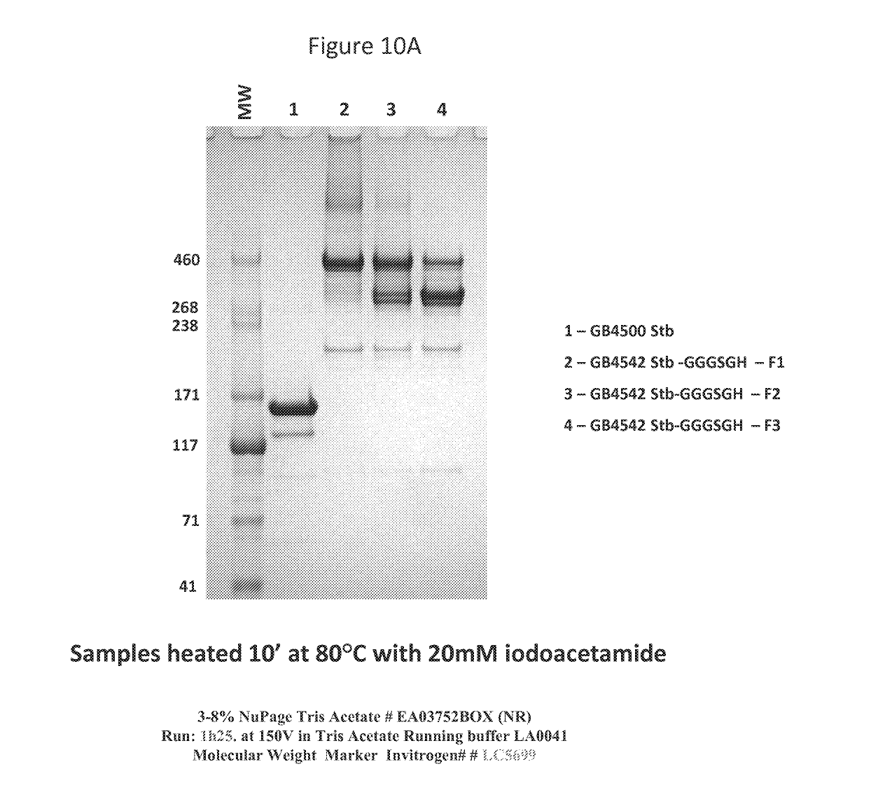

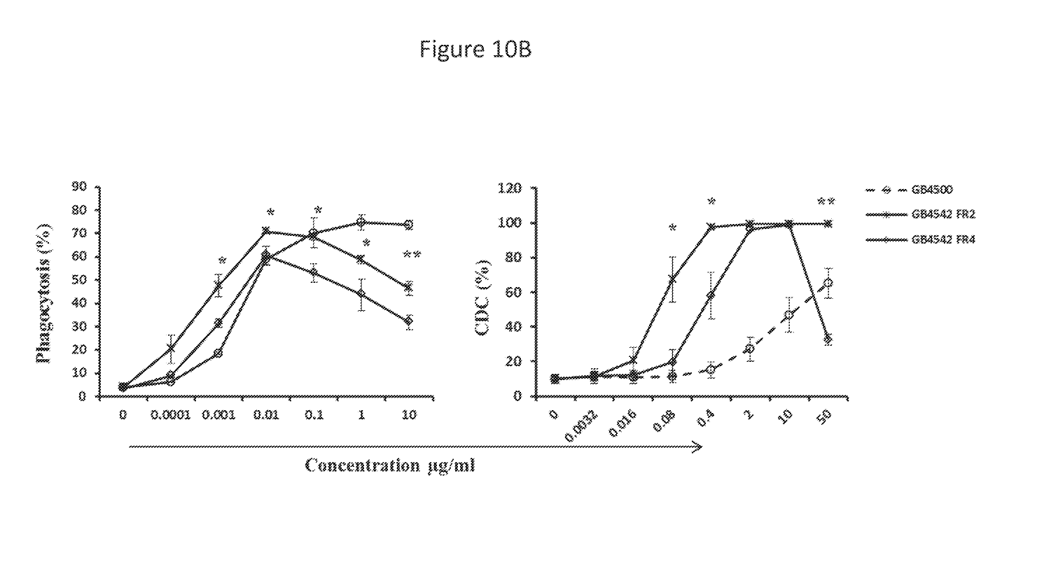

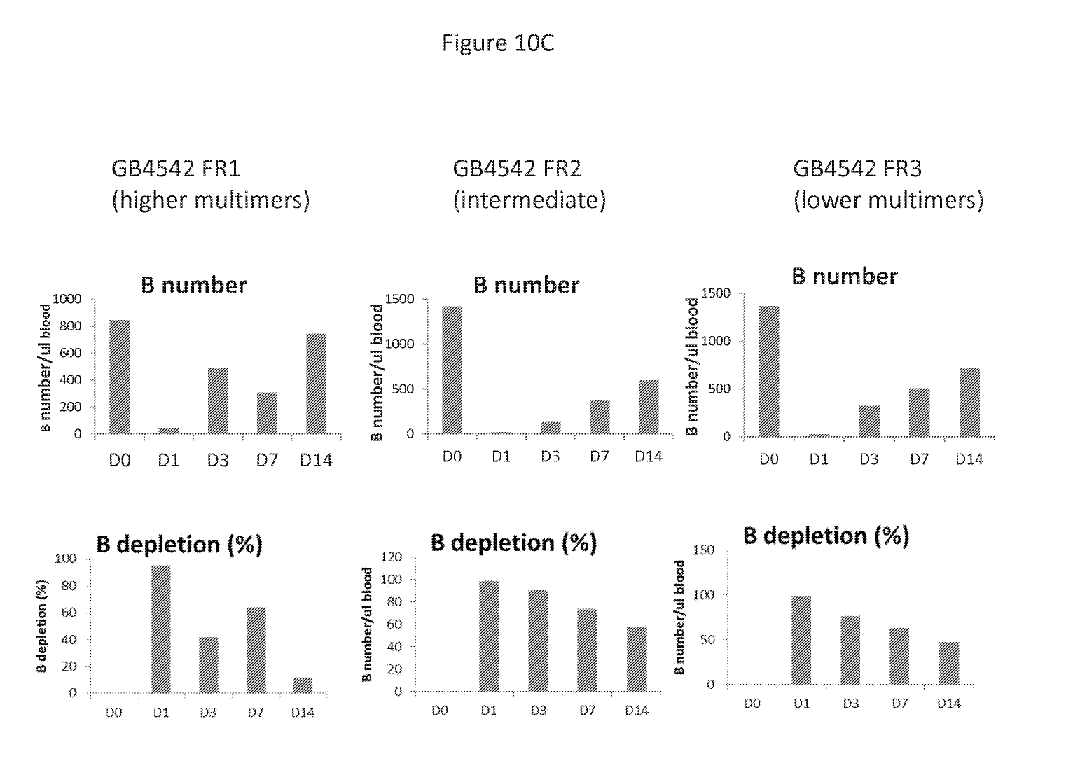

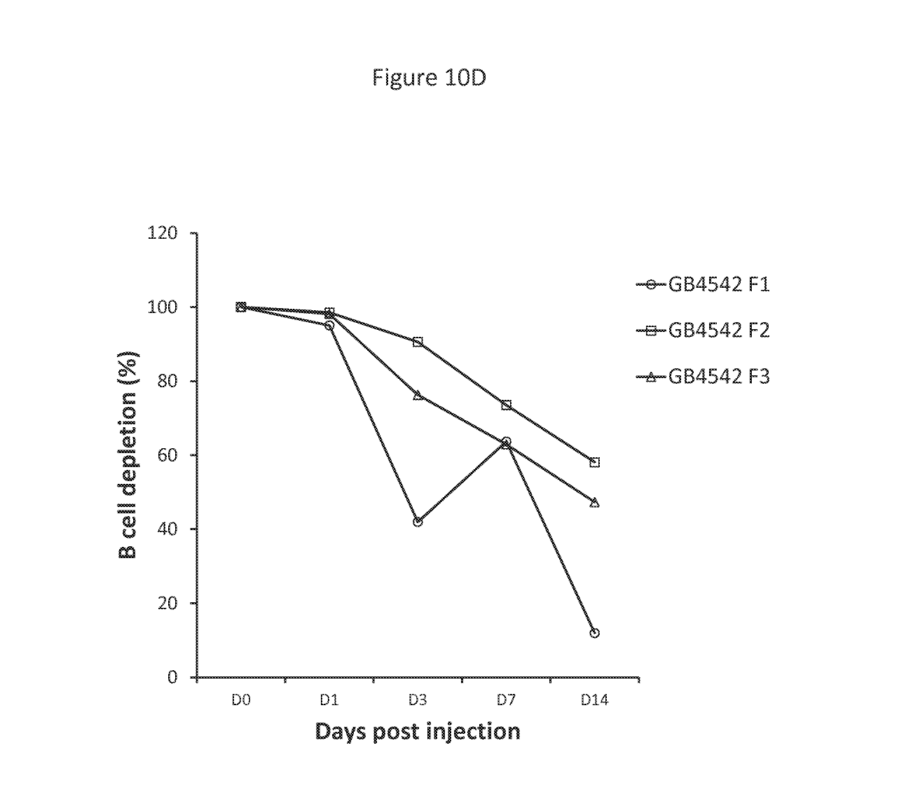

The higher and lower molecular weight fractions were separated to determine if higher order multimers exhibit different activity with respect to B cell depletion relative to lower order multimers. FIG. 10A shows the three GB4542 fractions (F1, F2, and F3, in lanes 2, 3, and 4, respectively) on a Coomassie gel.

To determine if there is a differential effect of higher and lower molecular weight fractions of GB4542, ADCP and CDC assays were conducted using FR2 and FR4 fractions of GB4542. FIG. 10B (left panel) shows that although both FR2 and FR4 exhibited concentration dependent bi-modal effects, FR2 mediated more potent phagocytic activity. FIG. 10B (right panel) shows that FR4 is less potent than FR2 in inducing CDC at low concentrations. In addition, and unlike FR2 or GB4500, FR4 inhibited CDC by approximately 80% at 50 μg/ml.

To determine if there is a differential effect of higher order and lower order molecular weight fractions in vivo, Cynomolgus monkeys were administered 1 mg/kg GB4542 fraction 1 (FR1), fraction 2 (FR2), or fraction 3 (FR3) at day 0 by subcutaneous injection. Blood was drawn at days 1, 3, 7, and 14 after treatment, and B cell depletion was analyzed by flow cytometry.

The results of the study are provided in FIGS. 10C and 10D. FIG. 10C is a set of bar graphs showing the total B cell number per μL blood and the percent depletion of B cells in the blood at days 0, 1, 3, 7, and 14 after the single administration of GB4542 FR1, FR2, or FR3. FIG. 10D is a line graph showing the percent depletion of B cells in the blood at days 0, 1, 3, 7, and 14 after the single administration of FR1, FR2, or FR3. The higher molecular weight fraction (FR1) mediated less effective B cell depletion relative to the lower molecular weight fractions (FR2 and FR3). Thus, the in vivo data correlated with the in vitro data provided herein wherein the higher molecular weight fractions mediated less potent ADCP and CDC.

### Example 6. Optimization of Multimerization Domains

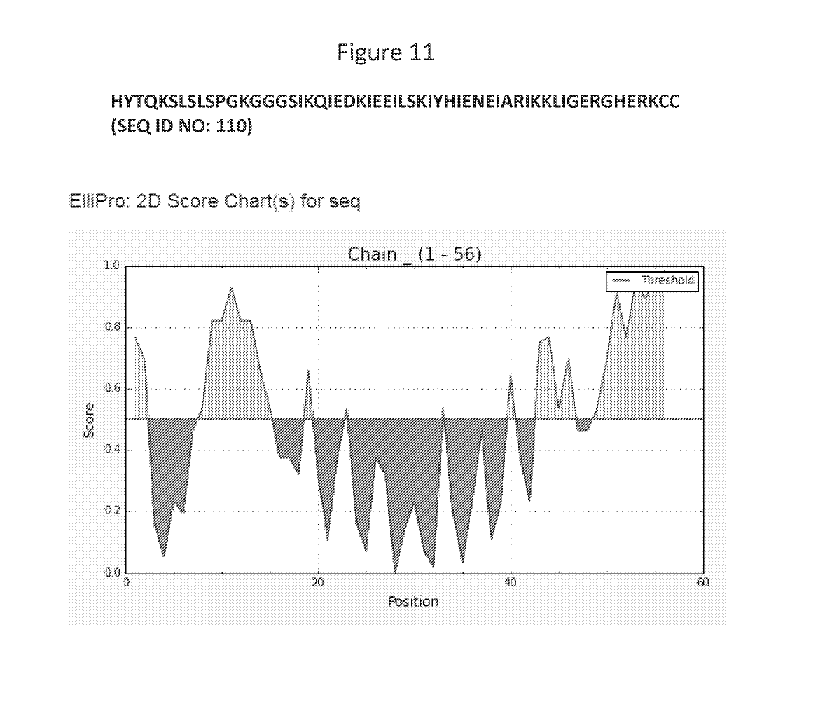

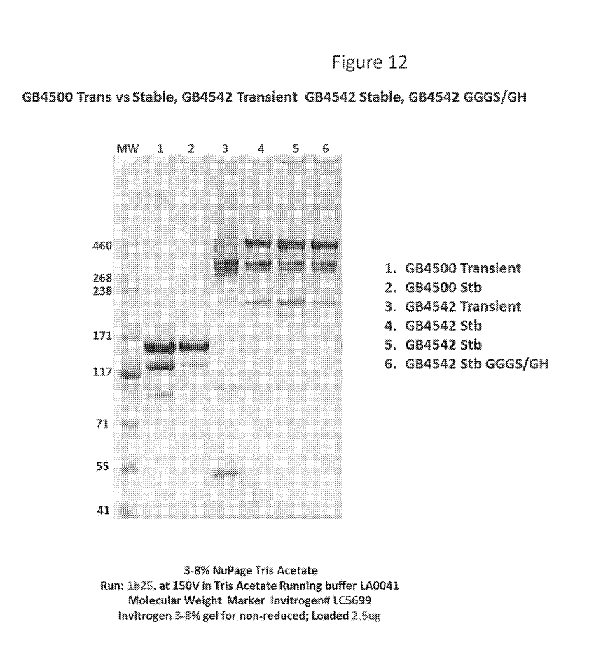

In silico analysis of the isoleucine zipper multimerization domain (SEQ ID NO: 99, contained in SEQ ID NO: 110) using an Ellipro test (Ponomarenko et al., BMC Bioinformatics 2008, 9:514) indicated that the sequence may be immunogenic (FIG. 11). Accordingly, stradobodies comprising modified multimerizing domain sequences were generated and the extent of multimerization was tested. The results are shown in FIG. 12. Lanes 1 and 2 are the GB4500 transient (expressed transiently) and GB4500 stable (expressed in a stable cell line pool) molecules. Lane 3 shows GB4542 having an amino acid sequence according to SEQ ID NO: 37 (comprising the unmodified multimerization domain sequence, SEQ ID NO: 32 and labeled as “GB4542 Transient”). Lanes 4, 5, show GB4542 comprising the amino acid sequences according to SEQ ID NO: 95 (comprising the multimerization domain sequence of SEQ ID NOs: 99. Lane 6 shows the GB4542 comprising the amino acid sequence according to SEQ ID 98 (comprising the multimerization domain of SEQ ID 102). The GB4542 transient (having the unmodified multimerization domain) has less high molecular weight banding relative to the stable GB4542 stradobodies having the modified multimerization domains. Thus, the stradobodies comprising the modified multimerization domains multimerize better than the stradobodies comprising the multimerization domain according to SEQ ID NO: 37 (comprising the multimerization domain of SEQ ID 32.

Taken together, the studies provided herein show that anti-CD20 stradobodies offer the ability to induce B cell depletion in subjects in vivo, and that at higher doses, continuous doses, or using higher order multimers, the stradobodies surprisingly and suppress cell killing. Thus, the stradobodies provided herein provide cell killing at low doses and IVIG-like tolerance at high doses; and provide cell killing when in lower order multimer form and IVIG-like tolerance when in higher order multimer form.

1. A method for inducing immune suppression comprising contacting an immune cell with a multimerizing stradobody.

2. The method of claim 1, wherein the immune cell is present in vitro, and wherein the stradobody is present at a concentration of more than about 1 μg/mL.

3. The method of claim 1, wherein the immune cell is present in a subject, and wherein the stradobody is administered to the subject at a dose level of more than about 1 mg/kg.

4. The method of claim 1, wherein the stradobody comprises an Fab domain, at least one multimerization domain, and at least one Fc domain.

5. The method of claim 4, wherein the stradobody comprises an Fab domain, two Fc domains, an IgG2 hinge, and an isoleucine zipper.

6. The method of claim 4, wherein the at least one Fc domain is an IgG1 Fc domain.

7. The method of claim 5, wherein the IgG1 Fc domain comprises an IgG1 hinge, IgG1 CH2, and IgG1 CH3.

8. The method of claim 1, wherein the amino acid sequence of the stradobody is at least 80% homologous to a sequence selected from the group consisting of: SEQ ID NOs: 33, 35, 37, 66, 92, 95, 96, 97, 98, 104, and 108.

9. A method for inducing target cell depletion or followed by suppression of inflammation in a subject comprising administering to the subject a stradobody, wherein the stradobody comprises an Fab specific for a target antigen present on a target cell, and wherein the stradobody induces target cell depletion followed by suppression of inflammation.

10. The method of claim 9, wherein the suppression of inflammation occurs when the target cell depletion has reached optimal levels.

11. The method of claim 9, wherein the suppression of inflammation occurs when the target cell depletion has resulted in low or absent levels of target antigen.

12. A method for inducing target cell depletion followed by suppression of inflammation in a subject, the method comprising (i) administering a multimerizing stradobody at a first dose level, wherein the first dose level results in target cell depletion; and (ii) administering the multimerizing stradobody at a second dose level, wherein the second dose level is higher than the first dose level, and wherein the second dose level results in suppression of inflammation in the subject.

13. The method of claim 12, wherein the suppression of inflammation in the subject is measured by a reduction in inflammatory cytokines such as TNF-α and/or increase in anti-inflammatory cytokines such as IL-1RA and/or changes in cell populations such as an increase in Regulatory T cells and/or by changes in immune cell surface markers such as monocyte HLA-DR or B cell maturation markers and/or changes in complement components detectable in serum.

14. The method of claim 12, wherein the first dose level achieves optimal depletion of the target cell population.

15. The method of claim 12, wherein the first dose level is less than about 1 mg/kg.

16. The method of claim 12, wherein the second dose level is more than about 10 mg/kg.

17. The method of claim 12, wherein the stradobody comprises an Fab domain, at least one multimerization domain, and at least one Fc domain.

18. The method of claim 17, wherein the stradobody comprises an Fab domain, two Fc domains, an IgG2 hinge, and an isoleucine zipper.

19. The method of claim 17, wherein the at least one Fc domain is an IgG1 Fc domain.

20. The method of claim 19, wherein the IgG1 Fc domain comprises an IgG1 hinge, IgG1 CH2, and IgG1 CH3.

21. The method of claim 12, wherein the stradobody comprises an Fab domain specific for CD20, EGFR, TNFα, Rho(D), HER2/neu, IL17, or IL12/23.

22. The method of claim 21, wherein the stradobody comprises an Fab domain that is specific for CD20, and wherein the first dose level induces depletion of B cells in the subject.

23. The method of claim 12, wherein the amino acid sequence of the stradobody is at least 80% homologous to a sequence selected from the group consisting of: SEQ ID NOs: 33, 35, 37, 66, 92, 95, 96, 97, 98, 104, and 108.

24. The method of claim 12, wherein the subject is a human.

25. A method for treating a disease or condition in a subject in need thereof, the method comprising administering a multimerizing stradobody to the subject at a first dose level followed by a second dose level, wherein the stradobody comprises an Fab domain specific for an antigen expressed on a target immune cell, cancer cell, or infectious agent that is present in the subject.

26. The method of claim 25, wherein the second dose level is higher than the first dose level.

27. The method of claim 25, wherein the first dose level induces target cell depletion in the subject.

28. The method of claim 25, wherein the second dose level induces suppression of inflammation in the subject.

29. The method of claim 25, wherein the first dose level is less than about 1 mg/kg.

30. The method of claim 25, wherein the second dose level is more than about 10 mg/kg.

31. The method of claim 25, wherein the stradobody comprises an Fab domain, at least one multimerization domain, and at least one Fc domain.

32. The method of claim 31, wherein the stradobody comprises an Fab domain, two Fc domains, an IgG2 hinge, and an isoleucine zipper.

33. The method of claim 32, wherein the at least one Fc domain is an IgG1 Fc domain.

34. The method of claim 33, wherein the IgG1 Fc domain comprises an IgG1 hinge, IgG1 CH2, and IgG1 CH3.

35. The method of claim 25, wherein the stradobody comprises an Fab domain specific for CD20, EGFR, TNFα, Rho(D), HER2/neu, IL17, or IL12/23.

36. The method of claim 35, wherein the stradobody comprises an Fab domain that is specific for CD20, and wherein the first dose level induces depletion of B cells in the subject.

37. The method of claim 25, wherein the amino acid sequence of the stradobody is at least 80% homologous to a sequence selected from the group consisting of: SEQ ID NOs: 33, 35, 37, 66, 92, 95, 96, 97, 98, 104, and 108.

38. The method of claim 25, wherein the subject is a human.

39. The method of claim 25, wherein the disease or condition is an inflammatory disease, autoimmune disease, infectious disease, or cancer.

40. The method of claim 39, wherein the stradobody comprises an Fab domain specific for CD20, and wherein the cancer is a B cell cancer.

41. The method of claim 25, wherein the first dose level induces ADCC, ADCP, CDC, or a combination thereof.

42. The method of claim 25, wherein the first dose level induces inflammatory cytokine production.

43. The method of claim 25, wherein the second dose level inhibits inflammatory cytokine production.

44. A method for treating a subject having a disease caused by an infectious agent, the method comprising administering to the subject a stradobody, wherein the stradobody comprises an Fab specific for a target antigen on the infectious agent, and wherein the stradobody induces opsonization and destruction of the infectious agent followed by suppression of inflammation.

45. A method for inducing destruction of an infectious agent followed by suppression of inflammation in a subject, the method comprising (i) administering to the subject a stradobody comprising an Fab domain that is specific for an antigen on the infectious agent at a first dose level, wherein the first dose level results in the opsonization and destruction of the infectious agent; and (ii) administering to the subject the stradobody at a second dose level, wherein the second dose level is higher than the first dose level, and wherein the second dose level results in suppression of inflammation in the subject.

46. The method of claim 1, wherein the stradobody is a higher order multimer.

47. The method of claim 46, wherein the stradobody is comprised of more than about 50% multimer bands at higher orders than the homodimer and dimer of the homodimer.

48. A method for inducing target cell depletion followed by suppression of inflammation in a subject, the method comprising (i) administering a first multimerizing stradobody that is a homodimer or a lower order multimer, wherein the first multimerizing stradobody results in target cell depletion; and (ii) administering a second multimerizing stradobody, wherein the second multimerizing stradobody is a higher order multimer, and wherein the second multimerizing stradobody results in suppression of inflammation in the subject.

49. The method of claim 48, wherein the suppression of inflammation in the subject is measured by a reduction in inflammatory cytokines such as TNF-α and/or increase in anti-inflammatory cytokines such as IL-1RA and/or changes in cell populations such as an increase in Regulatory T cells and/or by changes in immune cell surface markers such as monocyte HLA-DR or B cell maturation markers and/or changes in complement components detectable in serum.

50. The method of claim 48, wherein the administration of the first multimerizing stradobody achieves optimal depletion of the target cell population.

51. The method of claim 48, wherein the first and second multimerizing stradobodies each comprise an Fab domain, at least one multimerization domain, and at least one Fc domain.

52. The method of claim 51, wherein the first and second multimerizing stradobodies each comprise an Fab domain, two Fc domains, an IgG2 hinge, and an isoleucine zipper.

53. The method of claim 51, wherein the at least one Fc domain is an IgG1 Fc domain.

54. The method of claim 53, wherein the IgG1 Fc domain comprises an IgG1 hinge, IgG1 CH2, and IgG1 CH3.

55. The method of claim 48, wherein the first and second multimerizing stradobodies each comprise an Fab domain specific for CD20, EGFR, TNFα, Rho(D), HER2/neu, IL17, or IL12/23.

56. The method of claim 55, wherein the Fab domain is specific for CD20, and wherein the first multimerizing stradobody induces depletion of B cells in the subject.

57. The method of claim 48, wherein the amino acid sequence of each of the first and second multimerizing stradobodies is at least 80% homologous to a sequence selected from the group consisting of: SEQ ID NOs: 33, 35, 37, 66, 92, 95, 96, 97, 98, 104, and 108.

58. The method of claim 48, wherein the subject is a human.

59. A method for treating a disease or condition in a subject in need thereof, the method comprising administering a first multimerizing stradobody to the subject followed by a second multimerizing stradobody, wherein the first multimerizing stradobody is a homodimer or a lower order multimer, wherein the second stradobody is a higher order multimer, and wherein each of the first and second stradobodies comprises an Fab domain specific for an antigen expressed on a target immune cell, cancer cell, or infectious agent that is present in the subject.

60. The method of claim 59, wherein the first multimerizing stradobody induces target cell depletion in the subject.

61. The method of claim 59, wherein the second multimerizing stradobody induces suppression of inflammation in the subject.

62. The method of claim 59, wherein the first multimerizing stradobody induces ADCC, ADCP, CDC, or a combination thereof.

63. The method of claim 59, wherein the first multimerizing stradobody induces inflammatory cytokine production.

64. The method of claim 59, wherein the second multimerizing stradobody inhibits inflammatory cytokine production.

65. The method of claim 59, wherein the first and second multimerizing stradobodies each comprise an Fab domain, at least one multimerization domain, and at least one Fc domain.

66. The method of claim 65, wherein the first and second multimerizing stradobodies each comprise an Fab domain, two Fc domains, an IgG2 hinge, and an isoleucine zipper.

67. The method of claim 65, wherein the at least one Fc domain is an IgG1 Fc domain.

68. The method of claim 67, wherein the IgG1 Fc domain comprises an IgG1 hinge, IgG1 CH2, and IgG1 CH3.

69. The method of claim 59, wherein the first and second multimerizing stradobodies each comprise an Fab domain specific for CD20, EGFR, TNFα, Rho(D), HER2/neu, IL17 or IL12/23.

70. The method of claim 69, wherein the Fab domain is specific for CD20, and wherein the first multimerizing stradobody induces depletion of B cells in the subject.

71. The method of claim 59, wherein the amino acid sequence of each of the first and second multimerizing stradobodies is at least 80% homologous to a sequence selected from the group consisting of: SEQ ID NOs: 33, 35, 37, 66, 92, 95, 96, 97, 98, 104, and 108.

72. The method of claim 59, wherein the subject is a human.

73. The method of claim 59, wherein the disease or condition is an inflammatory disease, autoimmune disease, infectious disease, or cancer.

74. A method for inducing destruction of an infectious agent followed by suppression of inflammation in a subject, the method comprising (i) administering to the subject a first stradobody comprising an Fab domain that is specific for an antigen on the infectious agent, wherein the first stradobody is a homodimer or a lower order multimer and wherein the administration of the first stradobody results in the opsonization and destruction of the infectious agent; and (ii) administering to the subject a second stradobody comprising an Fab domain that is specific for the antigen on the infectious agent, wherein the second stradobody is a higher order multimer and wherein the administration of the second stradobody results in suppression of inflammation in the subject.

75. A method for treating cancer or an infectious disease in a subject, the method comprising administering a multimerizing stradobody to the subject, wherein the multimerizing stradobody is a homodimer or a lower order multimer, and wherein the multimerizing stradobody comprises an Fab domain specific for an antigen expressed on a tumor or a cancer cell or on an infectious agent.

76. The method of claim 75, wherein the Fab domain is specific for HER2/neu, EGFR, or CD20.

77. A method for treating cancer or an infectious disease in a subject, the method comprising administering a multimerizing stradobody to the subject, wherein the multimerizing stradobody comprises an Fab domain specific for an antigen expressed on a tumor or a cancer cell or on an infectious agent, and wherein the multimerizing stradobody is administered at a dose level of less than about 1 mg/kg.

78. The method of claim 77, wherein the Fab domain is specific for HER2/neu, EGFR, or CD20.


SEQUENCE LISTING (114 sequences)

SEQ ID NO: 1 - protein, 20 aa, Homo sapiens
METDTLLLWVLLLWVPGSTG

SEQ ID NO: 2 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 12 aa, Homo sapiens
ERKCCVECPPCP

SEQ ID NO: 4 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 5 - protein, 41 aa, Homo sapiens
GGGSIKQIEDKIEEILSKIYHIENEIARIKKLIGERGHGGG

SEQ ID NO: 6 - protein, 245 aa, Homo sapiens
DAADIQHSGGRSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 260 aa, Homo sapiens
DAADIQHSGGRSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEERKCCVECPPCP

SEQ ID NO: 8 - protein, 264 aa, Homo sapiens
METDTLLLWVLLLWVPGSTGERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 294 aa, Homo sapiens
METDTLLLWVLLLWVPGSTGGGSIKQIEDKIEEILSKIYHIENEIARIKKLIGERGHGGGSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 289 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSIKQIEDKIEEILSKIYHIENEIARIKKLIGERG

SEQ ID NO: 11 - protein, 266 aa, Mus sp.
METDTLLLWVLLLWVPGSTGDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKERKCCVECPPCP

SEQ ID NO: 12 - protein, 278 aa, Mus sp.
METDTLLLWVLLLWVPGSTGDAADIQHSGGRSREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKERKCCVECPPCP

SEQ ID NO: 13 - protein, 265 aa, Mus sp.
METDTLLLWVLLLWVPGSTGERKCCVECPPCPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 287 aa, Mus sp.
METDTLLLWVLLLWVPGSTGSIKQIEDKIEEILSKIYHIENEIARIKKLIGERGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 291 aa, Mus sp.
METDTLLLWVLLLWVPGSTGDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGSIKQIEDKIEEILSKIYHIENEIARIKKLIGERG

SEQ ID NO: 16 - protein, 299 aa, Mus sp.
METDTLLLWVLLLWVPGSTGDAADIQHSGGRSSIKQIEDKIEEILSKIYHIENEIARIKKLIGERGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - DNA, 753 bp, Homo sapiens
atggagacagacacactcctgctatgggtactgctgctctgggttccaggttccactggtgagcgcaaatgttgtgtggagtgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataa

SEQ ID NO: 18 - protein, 249 aa, Homo sapiens
METDTLLLWVLLLWVPGSTGERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 21 - protein, 264 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYAVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 22 - protein, 266 aa, Mus sp.
METDTLLLWVLLLWVPGSTGDEPRGPTIKPCPPCKCPAPNLLGGPSVFIFPPKIKDVLMISLSPIVTCVVVDVSEDDPDVQISWFVNNVEVHTAQTQTHREDYNATLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKERKCCVECPPCP

SEQ ID NO: 23 - protein, 266 aa, Mus sp.
METDTLLLWVLLLWVPGSTGDEPRGPTIKPCPPCKCPAPNLLGGPSVFIFPPKIKDVLMISLSPIVTCVVVDVSEDDPDVQISWFVNNVEVHTAQTQTHREDYNSTLAVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKERKCCVECPPCP

SEQ ID NO: 24 - protein, 282 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGPPGPPGPPGPPGPPGPPGPPGPPGPPGPP

SEQ ID NO: 25 - protein, 284 aa, Mus sp.
METDTLLLWVLLLWVPGSTGDEPRGPTIKPCPPCKCPAPNLLGGPSVFIFPPKIKDVLMISLSPIVTCVVVDVSEDDPDVQISWFVNNVEVHTAQTQTHREDYNATLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGPPGPPGPPGPPGPPGPPGPPGPPGPPGPP

SEQ ID NO: 26 - protein, 30 aa, Artificial Sequence
  GPP multimerization domain
GPPGPPGPPGPPGPPGPPGPPGPPGPPGPP

SEQ ID NO: 27 - protein, 282 aa, Homo sapiens
METDTLLLWVLLLWVPGSTGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGPPGPPGPPGPPGPPGPPGPPGPPGPPGPP

SEQ ID NO: 28 - protein, 282 aa, Homo sapiens
METDTLLLWVLLLWVPGSTGGPPGPPGPPGPPGPPGPPGPPGPPGPPGPPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 284 aa, Mus sp.
METDTLLLWVLLLWVPGSTGDEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGPPGPPGPPGPPGPPGPPGPPGPPGPPGPP

SEQ ID NO: 30 - protein, 283 aa, Mus sp.
METDTLLLWVLLLWVPGSTGGPPGPPGPPGPPGPPGPPGPPGPPGPPGPPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 217 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 32 - protein, 40 aa, Homo sapiens
GGGSIKQIEDKIEEILSKIYHIENEIARIKKLIGERGHDI

SEQ ID NO: 33 - protein, 764 aa, Homo sapiens [sequence omitted]

SEQ ID NO: 34 - protein, 218 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 35 - protein, 765 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCPRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 219 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 37 - protein, 766 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCPRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 470 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 234 aa, Homo sapiens
METDTLLLWVLLLWVPGSTGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 42 - protein, 497 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPKSCDKTHTCPPCPERKCCVECPPCP

SEQ ID NO: 43 - protein, 467 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 512 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGPPGPPGPPGPPGPPGPPGPPGPPGPPGPPERKCCVECPPCP

SEQ ID NO: 45 - protein, 512 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGPPGPPGPPGPPGPPGPPGPPGPPGPPGPPERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 729 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 754 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSVEGGGSIKQIEDKIEEILSKIYHIENEIARIKKLIGERGHGGGRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 725 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEERKCCVECPPCPRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 719 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 710 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 471 aa, Mus sp.
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 467 aa, Mus sp.
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 53 - protein, 451 aa, Homo sapiens
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 213 aa, Homo sapiens
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 471 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 233 aa, Homo sapiens
METDTLLLWVLLLWVPGSTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 468 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 472 aa, Mus sp.
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 59 - protein, 468 aa, Mus sp.
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 60 - protein, 449 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 213 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 62 - protein, 469 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 233 aa, Homo sapiens
METDTLLLWVLLLWVPGSTGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 64 - protein, 471 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 234 aa, Homo sapiens [sequence omitted]

SEQ ID NO: 66 - protein, 766 aa, Homo sapiens [sequence omitted]

SEQ ID NO: 67 - protein, 219 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 68 - protein, 527 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCPRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 513 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSIKQIEDKIEEILSKIYHIENEIARIKKLIGERGHDIERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 524 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCP

SEQ ID NO: 71 - protein, 496 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPKSCDKTHTCPPCPERKCCVECPPCP

SEQ ID NO: 72 - protein, 511 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGPPGPPGPPGPPGPPGPPGPPGPPGPPGPPERKCCVECPPCP

SEQ ID NO: 73 - protein, 728 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 753 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSVEGGGSIKQIEDKIEEILSKIYHIENEIARIKKLIGERGHGGGRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 713 aa, Homo sapiens
METDTLLLWVLLLWVPGSTGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 526 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCP

SEQ ID NO: 77 - protein, 498 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPKSCDKTHTCPPCPERKCCVECPPCP

SEQ ID NO: 78 - protein, 513 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGPPGPPGPPGPPGPPGPPGPPGPPGPPGPPERKCCVECPPCP

SEQ ID NO: 79 - protein, 730 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 755 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSVEGGGSIKQIEDKIEEILSKIYHIENEIARIKKLIGERGHGGGRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 715 aa, Homo sapiens [sequence omitted]

SEQ ID NO: 82 - protein, 498 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPKSCDKTHTCPPCPERKCCVECPPCP

SEQ ID NO: 83 - protein, 513 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGPPGPPGPPGPPGPPGPPGPPGPPGPPGPPERKCCVECPPCP

SEQ ID NO: 84 - protein, 730 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 755 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSVEGGGSIKQIEDKIEEILSKIYHIENEIARIKKLIGERGHGGGRLEGPRFEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 715 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 526 aa, Homo sapiens
METDTLLLWVLLLWVPGSTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCP

SEQ ID NO: 88 - protein, 729 aa, Homo sapiens; identical to SEQ ID NO: 46

SEQ ID NO: 89 - protein, 714 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 754 aa, Homo sapiens; identical to SEQ ID NO: 47

SEQ ID NO: 91 - protein, 525 aa, Homo sapiens
METDTLLLWVLLLWVPGSTGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSLEGGGSIKQIEDKIEEILSKIYHIENEIARIKKLIGERGHDIERKCCVECPPCP

SEQ ID NO: 92 - protein, 757 aa, Homo sapiens
METDTLLLWVLLLWVPGSTGQVKLLESGGGVVQPGGSLRVACVASGFTFRNFGMHWVRQAPGKGLEWVAFIWFDASNKGYGDSVKGRFTVSRDNSKNTLYLQMNGLRAEDTAVYYCAREKAVRGISRYNYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSIKQIEDKIEEILSKIYHIENEIARIKKLIGERGHERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 475 aa, Homo sapiens
METDTLLLWVLLLWVPGSTGQVKLLESGGGVVQPGGSLRVACVASGFTFRNFGMHWVRQAPGKGLEWVAFIWFDASNKGYGDSVKGRFTVSRDNSKNTLYLQMNGLRAEDTAVYYCAREKAVRGISRYNYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 232 aa, Homo sapiens
METDTLLLWVLLLWVPGSTGVMTQSPSSLSASVGDRVTITCRASQSIIRYLNWYQHKPGKAPKLLIHTASSLQSGVPSRFSGSVSGTDFTLTISSLQPEDFATYYCQQSYTTPYTFGQGTKLQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 753 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSIKQIEDKIEEILSKIYHIENEIARIKKLIGERGHERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 749 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIKQIEDKIEEILSKIYHIENEIARIKKLIGERGHERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 751 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSIKQIEDKIEEILSKIYHIENEIARIKKLIGERERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 747 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIKQIEDKIEEILSKIYHIENEIARIKKLIGERERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 38 aa, Homo sapiens
GGGSIKQIEDKIEEILSKIYHIENEIARIKKLIGERGH

SEQ ID NO: 100 - protein, 34 aa, Homo sapiens
IKQIEDKIEEILSKIYHIENEIARIKKLIGERGH

SEQ ID NO: 101 - protein, 36 aa, Homo sapiens
GGGSIKQIEDKIEEILSKIYHIENEIARIKKLIGER

SEQ ID NO: 102 - protein, 32 aa, Homo sapiens
IKQIEDKIEEILSKIYHIENEIARIKKLIGER

SEQ ID NO: 103 - protein, 477 aa, Homo sapiens
METDTLLLWVLLLWVPGSTGEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 759 aa, Homo sapiens
METDTLLLWVLLLWVPGSTGEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSIKQIEDKIEEILSKIYHIENEIARIKKLIGERGHERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 235 aa, Homo sapiens
METDTLLLWVLLLWVPGSTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 469 aa, Homo sapiens Homo sapiens
METDTLLLWVLLLWVPGSTGEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 234 aa, Homo sapiens
METDTLLLWVLLLWVPGSTGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 751 aa, Homo sapiens
METDTLLLWVLLLWVPGSTGEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSIKQIEDKIEEILSKIYHIENEIARIKKLIGERGHERKCCVECPPCPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 234 aa, Homo sapiens [sequence omitted]

SEQ ID NO: 110 - protein, 56 aa, Homo sapiens
HYTQKSLSLSPGKGGGSIKQIEDKIEEILSKIYHIENEIARIKKLIGERGHERKCC

SEQ ID NO: 111 - protein, 483 aa, Homo sapiens
METDTLLLWVLLLWVPGSTGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 112 - protein, 487 aa, Homo sapiens
METDTLLLWVLLLWVPGSTGQVKLLESGGGVVQPGGSLRVACVASGFTFRNFGMHWVRQAPGKGLEWVAFIWFDASNKGYGDSVKGRFTVSRDNSKNTLYLQMNGLRAEDTAVYYCAREKAVRGISRYNYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 113 - protein, 489 aa, Homo sapiens
METDTLLLWVLLLWVPGSTGEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP

SEQ ID NO: 114 - protein, 481 aa, Homo sapiens
METDTLLLWVLLLWVPGSTGEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKERKCCVECPPCP